(19) United States
(12) Patent Application Publication
(10) Pub. No.: US 2013/0316388 A1
(43) Pub. Date: Nov. 28, 2013

(54) PROCESS AND APPARATUS FOR RAPID, HIGH-THROUGHPUT ANALYSIS OF FATTY ACIDS

(71) Applicant: HEALTH DIAGNOSTIC LABORATORY, INC., Richmond, VA (US)

(72) Inventors: James BOROWSKI, Richmond, VA (US); Alexandra SHERMAN, Richmond, VA (US); Jennifer WARD, Richmond, VA (US); Joseph P. McCONNELL, Richmond, VA (US)

(73) Assignee: HEALTH DIAGNOSTIC LABORATORY, INC., Richmond, VA (US)

(21) Appl. No.: 13/827,447

(22) Filed: Mar. 14, 2013

Related U.S. Application Data
(60) Provisional application No. 61/651,987, filed on May 25, 2012.
(60) Provisional application No. 61/696,011, filed on Aug. 31, 2012.
(60) Provisional application No. 61/696,613, filed on Sep. 4, 2012.

(51) Int. Cl. G01N 33/92

(52) U.S. Cl. CPC G01N 33/92
USPC 435/29; 436/45; 435/294.1; 435/288.7; 422/70; 422/89

(57) ABSTRACT

This invention relates to an apparatus and a process for rapid, high-throughput analysis of fatty acids in a plurality of samples. The apparatus comprises at least one multi-vessel plate, wherein each vessel is a unit for holding a sample, or mixing and/or reacting a sample with one or more solvents or reagents; at least one matching multi-cap mat capable of sealing the vessels of the multi-vessel plate during the holding, mixing and/or reacting the sample; at least one multi-vessel plate holder having sealing units, whereby the multi-vessel plate holder, when the sealing units are engaged, presses the matching multi-cap mat onto the tops of the vessels in the multi-vessel plate sealing the vessels, so as to withstand high pressure and high temperature conditions. The process employs the apparatus that enables automated, high-throughput analysis of twenty-four fatty acid from a plurality of samples by gas chromatography flame ionization detection.

### CROSS-REFERENCE TO RELATED APPLICATIONS

This application claims the benefit of priority to U.S. Provisional Patent Application Ser. No. 61/651,987, filed May 25, 2012; U.S. Provisional Patent Application Ser. No. 61/696,613, filed Sep. 4, 2012; and U.S. Provisional Patent Application Ser. No. 61/696,011, filed Aug. 31, 2012, all of which are hereby incorporated by reference in their entirety.

### FIELD OF THE INVENTION

This invention relates to an apparatus and a process for rapid, high-throughput analysis of one or more fatty acids in a plurality of samples.

### BACKGROUND

Fatty acids are essential constitutes of cells and play a variety of roles in cellular signaling, intercellular attachment, transport of molecules, identification of foreign material, etc. Fatty acids are of greatest importance to human nutrition. In the human body, fatty acids serve as energy sources, precursors of prostaglandins, components of cell membranes and myelinization of the central nervous system.

The fatty acid compositions and the proportion of specific fatty acids, i.e. the fatty acid distribution in cells and blood, can be associated with a wide variety of diseases and conditions, such as heart diseases, cancer and autoimmune diseases. See Schaeffer et al., Human Molecular Genetics, 2006, 15 (11): 1745-56. The detection and profiling of fatty acid compositions thus become increasingly valuable so that a rapid and routine determination of a fatty acid profile can be established as a regular tool for a medical diagnostic marker as well as a nutritional physiological marker.

Conventional fatty acid analysis method typically involves using of manual pipettes/liquid dispensers, and glass vials/tubes with screw-up/snapped-sealed cap, which demand laborious manual separation and analysis processes. These processes are time-consuming, and, because of the associated costs, are unsuitable for simultaneously analyzing a large number of samples. Additionally, these manual processes involve repetitive motion of liquid/sample extracting, transferring, and various handling, which not only add risk for sample mix-up and increase imprecision and inaccuracies, but also can expose the technician to toxic fluids and chemicals, as the reaction conditions can involve high temperature, high pressure and toxic solvents.

Therefore, there is a need in the art to develop a rapid, high throughput technique for improved analysis of fatty acids in a plurality of samples with high sensitivity and high accuracy. This invention answers this need.

### SUMMARY OF THE INVENTION

One aspect of this invention relates to an apparatus that comprises at least one multi-vessel plate. Each vessel is a unit for holding a sample, or mixing and/or reacting a sample with one or more solvents or reagents. The apparatus also comprises at least one matching multi-cap mat that is capable of sealing the vessels of the multi-vessel plate during the holding, mixing and/or reacting the sample, and at least one multi-vessel plate holder has sealing units. The multi-vessel plate holder, when the sealing units are engaged, presses the matching multi-cap mat onto the tops of the vessels in the multi-vessel plate sealing the vessels, so as to withstand high pressure and high temperature conditions. The apparatus may also contain an optional multi-vessel plate heating unit capable of pre-heating to a desirable temperature prior to the introduction of the vessels containing the samples, and an optional multi-vessel plate separating unit capable of separating one component from the others, if two or more components are present in the vessel in the multi-vessel plate.

Another aspect of the invention relates to an apparatus for high-throughput esterification of fatty acids. The apparatus comprises a multi-vessel plate. Each vessel is a unit for mixing and/or reacting a sample containing one or more fatty acids with one or more solvents or reagents. The apparatus also comprises matching multi-cap mat that is capable of sealing the vessels of the multi-vessel plate during the mixing and/or reacting the sample, and a multi-vessel plate holder having sealing units. The multi-vessel plate holder, when the sealing units are engaged, presses the matching multi-cap mat onto the tops of the vessels in the multi-vessel plate sealing the vessels, so as to withstand high pressure and high temperature conditions. The apparatus also contains a multi-vessel plate heating unit capable of pre-heating to a temperature desirable for esterification of the fatty acids prior to the introduction of the vessels containing the fatty acids, and a multi-vessel plate separating unit capable of separating the esterified fatty acid from the sample in the vessel of the multi-vessel plate.

Another aspect of the invention relates to a rapid, high-throughput process of analyzing one or more fatty acids in a plurality of samples. The method comprises introducing a plurality of samples containing one or more fatty acids into individual vessels in a multi-vessel plate; mixing an esterification agent with each sample in the multi-vessel plate to produce esterified fatty acids; contacting the multi-vessel plate with a multi-vessel plate pre-heated to an esterification temperature of 50 to 300° C.; separating the esterified fatty acids from each sample; and analyzing the esterified fatty acids from each sample by gas or liquid chromatography. Each vessel of the multi-vessel plate is sealed by a matching multi-cap mat.

The embodiments of the invention provide an apparatus for high-throughput analysis of fatty acids. The automated homogenization (e.g., the multi-vessel vortexer/mixer) and heating/cooling elements (e.g., the multi-vessel heating/cooling unit) hold the entire plate of multiple vessels rather than each sample vessel separately, which provide simultaneous and instantaneous homogenization and/or heating/cooling. The automated liquid handling device eliminates repetitive motion of manually liquid/sample extracting, transferring, and various handling, which not only eliminate risks for sample mix-up but also improves the accuracy of sample analysis. The multi-vessel plate, matching multi-cap mat, and matching multi-vessel plate holder also improves the simultaneously sealing of the multiple vessels so that they withstand high temperatures and high pressures, preventing or helping prevent samples from evaporation, contamination or toxication by exposure to human contact.

In a conventional analysis, a person was typically able to process 150 samples in an 8-hour shift with the manual extraction method. Many erroneous factors can be involved in the process, e.g., sample mix-up from moving sample tubes between sample racks, heating blocks, vortex mixers, and centrifuges; transferring extracted samples between sample tubes; or problems with placing the samples on detecting instruments. With the apparatus described here, many of these errors can be avoided. For example, one person can process 1000 samples in an 8-hour shift with limited sample mix-ups, accidents, injuries or errors.

In an exemplary embodiment, this rapid, high-throughput fatty acid analysis test measures twenty-four fatty acid methyl esters (FAME) from erythrocyte membranes by gas chromatography flame ionization detection (GCFID). The twenty-four fatty acids include trans-palmitoleic, trans-oleic, trans-linoleic, cis-palmitoleic, cis-oleic, cis-eicosenoic, cis-nervonic, α-linolenic, eicosapentaenoic, docosapentaenoic, docosahexaenoic, linoleic, γ-linolenic, arachidonic, eicosadienoic, dihomo-γ-linolenic, docasatetraenoic, docosapentaenoic, myristic, palmitic, behenic, lignoceric, arachidic, and stearic acid. The process eliminates, or almost completely eliminates, the manual manipulation of samples and solvents in the separation of fatty acids from erythrocyte membranes. This improves throughput and quality of sample analysis and prevents, or significantly reduces, sample contamination. The entire process for quantifications of these twenty-four fatty acids during the analysis step by gas or liquid chromatography can be carried out relatively quickly, for instance, in less than about 10 minutes, or less than 6 minutes. See e.g., FIG. 1.

Additional aspects, advantages and features of the invention are set forth in this specification, and will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIG. 1 is a graph showing the results of chromatographic separation of illustrative twenty-four fatty acid methyl esters (FAME) from erythrocyte membranes by gas chromatography flame ionization detection (GCFID) using the high-throughput process configured to run for 8.5 minutes. Twenty-four fatty acid species were identified in the chromatogram using an internal standard (C13:0 fatty acid). The field of view in the figure was reduced to enlarge the size of the peaks for clarity. The analysis step from sample injection to completion was within 8.5 minutes.

FIG. 2 is a scheme showing exemplary elements for an apparatus for high-throughput esterifying and analyzing fatty acids.

FIGS. 3A-3C are photographs showing the liquid handler deck for the Hamilton Microlab STAR. FIG. 3A shows the 96-unit automatic pipette heads and tips that connect to the pipette heads; FIG. 3B shows the 96-well plate; and FIG. 3C shows the reagent tough.

FIGS. 4A-4D are photographs showing various elements for the apparatus including the preheating aluminum blocks (FIG. 4A), aluminum blocks sealing clamps (FIG. 4B), aluminum block heater shakers (FIG. 4C), and aluminum block chiller (FIG. 4D).

FIGS. 5A-5B are photographs showing CapMat Vise (FIG. 5A) for sealing teflon cap mat covering the 96-vial (150 μL glass inserts) plate (FIG. 5B).

FIGS. 6A-6B are graphs showing exemplary results of chromatographic separation of illustrative twenty-four fatty acid methyl esters (FAME) from erythrocyte membranes by gas chromatography flame ionization detection (GCFID). FIG. 6A shows a high-throughput process completed in 6 minutes. The field of view in the figure focuses on the 3-minute window where peaks are evident. FIG. 6B shows a high-throughput process completed in 16 minutes. The time for completing the high-throughput process can vary by changing the temperature ramp rate and separation column length.

FIGS. 7A-7B are graphs showing results comparing an automated, high-throughput method and a manual method for omega-3 fatty acid (FA) analysis. The automated, high-throughput method is exemplified as “HDL” in the graph and the manual method is exemplified as “OQ” in the graph. FIG. 7A shows the result of linear regression plot of HDL omega-3 FA analysis relative to OQ omega-3 FA analysis. FIG. 7B shows the result of Bland Altman plot of HDL omega-3 FA analysis relative to OQ omega-3 FA analysis.

FIGS. 8A-8B are graphs showing results comparing an automated, high-throughput method and a manual method for omega-6 fatty acid (FA) analysis. The automated, high-throughput method is exemplified as “HDL” in the graph and the manual method is exemplified as “OQ” in the graph. FIG. 8A shows the result of linear regression plot of HDL omega-6 FA analysis relative to OQ omega-6 FA analysis. FIG. 8B shows the result of Bland Altman plot of HDL omega-6 FA analysis relative to OQ omega-6 FA analysis.

FIGS. 9A-9B are graphs showing results comparing an automated, high-throughput method and a manual method for cis-monounsaturated fatty acid (FA) analysis. The automated, high-throughput method is exemplified as “HDL” in the graph and the manual method is exemplified as “OQ” in the graph. FIG. 9A shows the result of linear regression plot of HDL cis-monounsaturated FA analysis relative to OQ cis-monounsaturated FA analysis. FIG. 9B shows the result of Bland Altman plot of HDL cis-monounsaturated FA analysis relative to OQ cis-monounsaturated FA analysis.

FIGS. 10A-10B are graphs showing results comparing an automated, high-throughput method and a manual method for saturated fatty acid (FA) analysis. The automated, high-throughput method is exemplified as “HDL” in the graph and the manual method is exemplified as “OQ” in the graph. FIG. 10A shows the result of linear regression plot of HDL saturated FA analysis relative to OQ saturated FA analysis. FIG. 10B shows the result of Bland Altman plot of HDL saturated FA analysis relative to OQ saturated FA analysis.

FIGS. 11A-11B are graphs showing results comparing an automated, high-throughput method and a manual method for trans fatty acid (FA) analysis. The automated, high-throughput method is exemplified as “HDL” in the graph and the manual method is exemplified as “OQ” in the graph. FIG. 11A shows the result of linear regression plot of HDL trans FA analysis relative to OQ trans FA analysis. FIG. 11B shows the result of Bland Altman plot of HDL trans FA analysis relative to OQ trans FA analysis.

FIGS. 12A-12B are graphs showing results comparing an automated, high-throughput method and a manual method for omega-3 fatty acid (FA) index analysis. The automated, high-throughput method is exemplified as “HDL” in the graph and the manual method is exemplified as “OQ” in the graph. FIG. 12A shows the result of linear regression plot of HDL omega-3 FA index analysis relative to OQ omega-3 FA index analysis. FIG. 12B shows the result of Bland Altman plot of HDL omega-3 FA index analysis relative to OQ omega-3 FA index analysis.

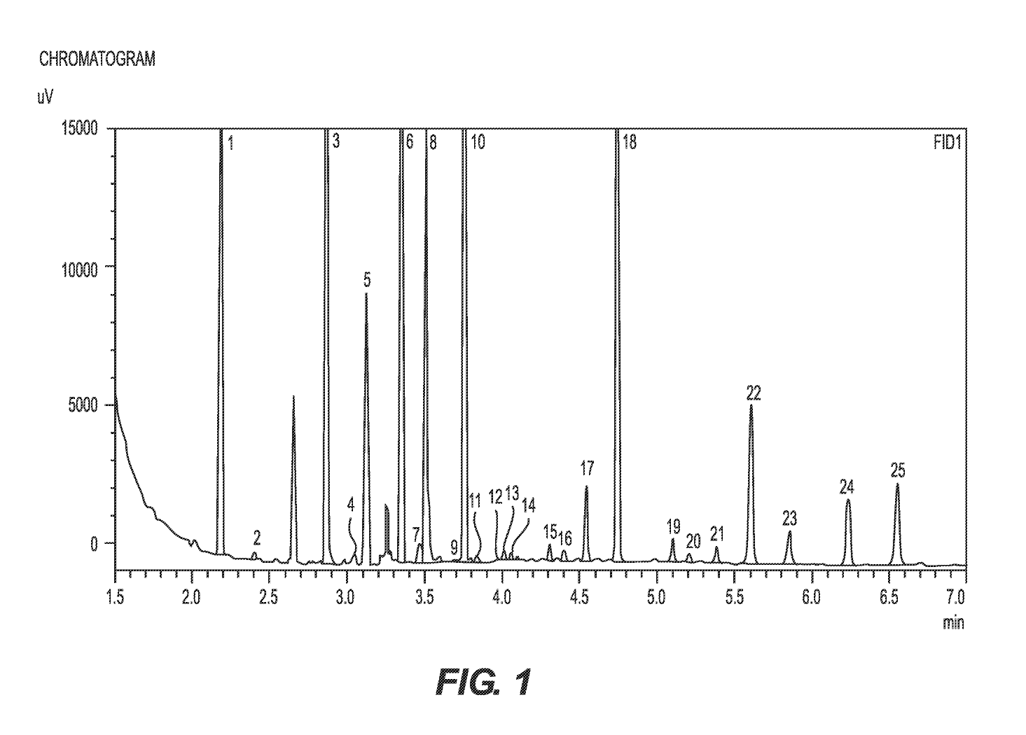

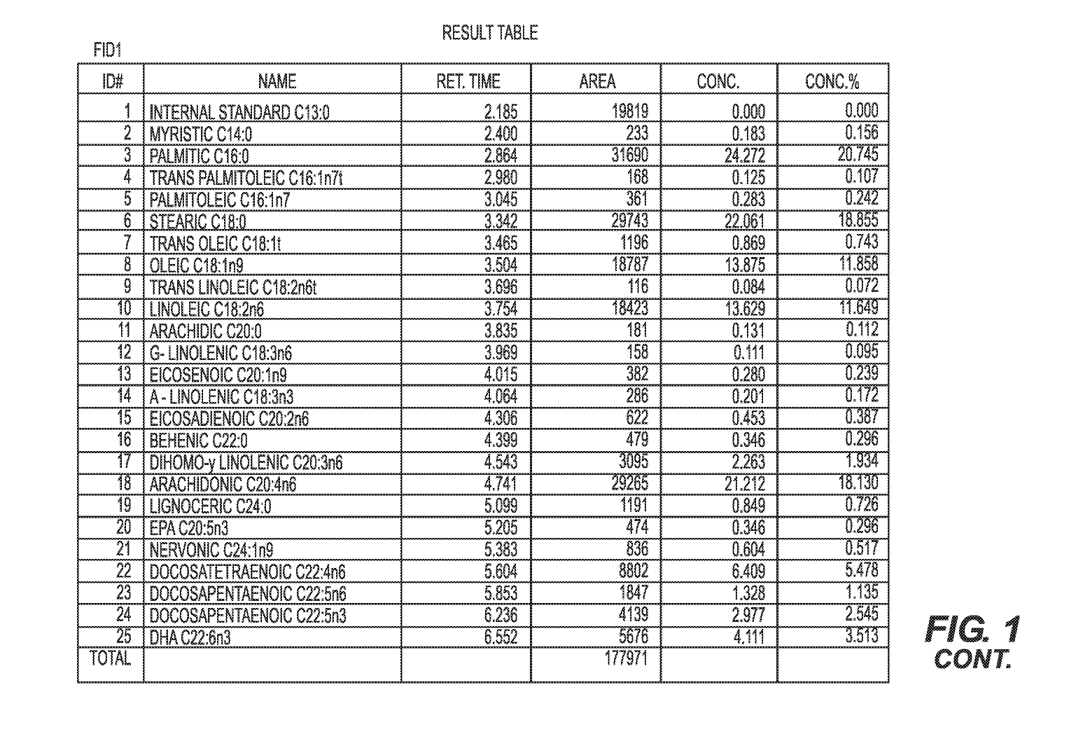

### DETAILED DESCRIPTION OF THE INVENTION

This invention relates to a process for rapid, high-throughput analysis of one or more fatty acids in a plurality of samples. The process employs a system or a novel apparatus that enables automated, high-throughput conduction of one or more steps of the process.

One aspect of this invention relates to an apparatus that comprises at least one multi-vessel plate. Each vessel is a unit for holding a sample, or mixing and/or reacting a sample with one or more solvents or reagents. The apparatus also comprises at least one matching multi-cap mat that is capable of sealing the vessels of the multi-vessel plate during the holding, mixing and/or reacting the sample, and at least one multi-vessel plate holder has sealing units. The multi-vessel plate holder, when the sealing units are engaged, presses the matching multi-cap mat onto the tops of the vessels in the multi-vessel plate sealing the vessels, so as to withstand high pressure and high temperature conditions. The apparatus may also contain an optional multi-vessel plate heating unit capable of pre-heating to a desirable temperature prior to the introduction of the vessels containing the samples, and an optional multi-vessel plate separating unit capable of separating one component from the others, if two or more components are present in the vessel in the multi-vessel plate.

This apparatus can include at least one multi-vessel plate. Each vessel of the multi-vessel plate is a unit for holding a sample, or mixing and/or reacting a sample with one or more solvents or reagents. Each vessel is wide and tall enough to allow for adequate mixing, and thin enough to allow the multi-vessel plate to fit in an automated fluid handling station and/or an automated multi-vessel plate handling station. The vessel can have a round or flat base depending on the requirement of the system.

The multi-vessel plate can have a matching multi-cap mat that is capable of sealing the vessels of the multi-vessel plate during the holding, mixing and/or reacting the sample. The lining of the multi-cap mat which contacts the tops of the vessels in the multi-vessel plate is made of a material that does not deteriorate and does not contaminate the vessel when heating to the desirable temperature. For instance, the material can be teflon. The thickness of the lining of the multi-cap mat can range from about 1 to about 10 mm; for instance, from about 4 to about 6 mm, or about 5 mm.

The use of multi-cap mat for the multi-vessel plate can reduce the time spent in screwing/unscrewing or snapping/unsnapping a cap to each vessel, particularly when a large number of samples are involved, and minimize risks of a glass vial cap blowing off or having the glass vial shatter. These advantages are particularly apparent when using the multi-vessel plate/multi-cap mat with a matching multi-vessel plate holder.

At least one multi-vessel plate holder that has a matching size with the multi-vessel plate can be used to hold the multi-vessel plate for temporary storage, or, during the holding, mixing and/or reacting the sample. The multi-vessel plate holder has sealing units, whereby the multi-vessel plate holder, when the sealing units are engaged, can press the matching multi-cap mat onto the tops of the vessels in the multi-vessel plate, effectively sealing the vessels to the point at which they can withstand high pressure and high temperature conditions.

The apparatus can optionally hold a library of stock multi-vessel plates, the plates having a variety of functions. For instance, they can be used to contain samples, react with reagents for certain reactions, or for extraction or separation of certain components in the samples, etc. Multi-vessel plates can be created as needed. For example, a first set of multi-vessel plates and its matching multi-cap mat can be used for processing the samples (including sample transferring, mixing, reacting, separating, etc.); and a second set of multi-vessel plates and its matching multi-cap mat can be used for holding and measuring the processed sample components transferred or separated from other components of the samples from the first set of multi-vessel plate. The size of vessel in different multi-vessel plate can vary in a wide range to fit the different needs. Each multi-vessel plate can have a matching multi-vessel plate holder.

An automated liquid/fluid handler (or an automated multi-vessel plate handler) can be used in the system. This automated liquid handling device can introduce weighed samples and/or reagents into each vessel. For instance, the automated liquid handling device may contain an automated pipetting device that is capable of automatedly pipetting a weighed amount of sample and/or solvent into each vessel. This automated liquid handling can reduce risks of inaccuracy and sample mix-up introduced from manual liquid handling and manually repetitive motion. Additionally, automated liquid/fluid handler can be placed in a location that human contact with toxic solvents are minimized.

The automated liquid handling device can optionally include one or more elements for automated homogenization (e.g., automated shaking, mixing, or vortexing), automated heating/cooling, and/or simultaneous automated homogenization and heating/cooling.

The optional heating/cooling element can be a multi-vessel plate heating/cooling unit capable of pre-heating/pre-cooling to a desired temperature prior to the introduction of the vessels containing the samples. Typically, the material of the multi-vessel plate heating unit is heat-conductive materials, for instance, a metal such as aluminum.

The optional automated homogenization element can be a multi-vessel plate mixer/shaker unit capable of mixing/shaking various samples in multiple vessels simultaneously. For instance, the multi-vessel plate mixer can be a multi-vessel plate vortexer.

The automated heating/cooling can be carried out on a separate multi-vessel plate heating/cooling unit. Similarly, the automated homogenization can be carried out on a separate multi-vessel plate shaking/mixing/vortexing unit.

Alternatively, the automated heating/cooling and homogenization elements can be combined in a same automated device. For instance, the multi-vessel plate heating unit can also be a multi-vessel plate mixer at the same time.

The apparatus can also include an optional multi-vessel plate separating unit capable of separating one component from the others, if two or more components are present in the vessel in the multi-vessel plate. For instance, the multi-vessel plate separating unit can be a multi-vessel plate centrifuge, capable of separating various samples in multiple vessels simultaneously. Alternatively, the apparatus may not have a separating unit, in which case the separation of components will proceed slowly over time.

The system/apparatus may further include equipment for labeling vessels in the multi-vessel plate and a label detector. For instance, the labeling equipment can be an automated bar-coding equipment, and the label detector can be an automated bar code detector. The labeling equipment and label detector can enable, for instance, the precise mapping of the measurements obtained for each sample in the vessel. The use of the labeling equipment can also minimize the risk of sample mix-up when manual numbering the sample vessels, particularly when large number of samples are involved.

The apparatus additionally includes a multi-vessel plate measuring unit to analyze the processed samples. The measuring unit enables automated quantization of each fatty acid (or esterified fatty acid) in the sample of each vessel. This measuring unit can be of modular construction, thereby permitting the different measuring units to be exchanged depending on the measurement task. Suitable measuring units include chromatography devices, such as a gas or liquid chromatography column. This measuring unit can further comprise a detector. The detector may include gas chromatography (GC)/mass spectrometry (MS), GC/MS/MS, liquid chromatography (LC)/MS, LC/MS/MS, GC/LC, GC/flame ionization detector (FID), high-performance liquid chromatography (HPLC), nuclear magnetic resonance (NMR), or similar chromatography systems and spectroscopic systems, such as NMR or fourier transform infrared spectroscopy (FTIR). For instance, the measuring unit can be a gas or liquid chromatography column with a mass spectrometry detector, an ionization detector or thermal conductivity detector. The system/apparatus can include an integrated robotic system having one or more robots or separate robotic units transporting the multi-vessel plates/mats/holders from station to station for sample and reagent addition, holding, mixing, incubation, and measurements.

The system/apparatus can also include data processing and control software. By means of an intelligent software program, the analysis of a plurality of samples may be optimized in terms of time, by conducting different steps in parallel when operating on batches of multi-vessel plates.

The apparatus is generally applicable for any sample handling for an automated process involving reactions where evaporation or contamination is a concern, or where the reaction conditions can be inferior or involves high pressure/temperature.

Particularly, the apparatus can be used for high-throughput esterifying and analyzing fatty acids. The samples to be processed in the apparatus are samples containing one or more fatty acids with one or more solvents or reagents for subsequent esterification and analysis. In this regard, the apparatus includes a multi-vessel plate heating unit which is used to pre-heat the sample to a temperature desirable for esterification of the fatty acids prior to the introduction of the vessels containing the fatty acids. The apparatus also includes a multi-vessel plate separating unit capable of separating the esterified fatty acid from the sample in the vessel of the multi-vessel plate.

Accordingly, embodiments of the invention provide an apparatus for the high-throughput esterification of fatty acids. The apparatus comprises a multi-vessel plate. Each vessel is a unit for mixing and/or reacting a sample containing one or more fatty acids with one or more solvents or reagents. The apparatus also comprises matching multi-cap mat that is capable of sealing the vessels of the multi-vessel plate during the mixing and/or reacting the sample, and a multi-vessel plate holder having sealing units. The multi-vessel plate holder, when the sealing units are engaged, presses the matching multi-cap mat onto the tops of the vessels in the multi-vessel plate sealing the vessels, so as to withstand high pressure and high temperature conditions. The apparatus also contains a multi-vessel plate heating unit capable of pre-heating to a temperature desirable for esterification of the fatty acids prior to the introduction of the vessels containing the fatty acids, and a multi-vessel plate separating unit capable of separating the esterified fatty acid from the sample in the vessel of the multi-vessel plate.

Embodiments for various elements in the apparatus described above for the general process also applies to the embodiments where the apparatus is used specifically for the high-throughput esterification and analysis of fatty acids.

The apparatus may further comprise a second multi-vessel plate for holding the separated esterified fatty acids from the plurality of samples; a second matching multi-cap mat capable of sealing the vessels of the second multi-vessel plate; and an optional multi-vessel plate holder having sealing units. The multi-vessel plate holder, when the sealing units are engaged, presses the matching multi-cap mat onto the tops of the vessels in the multi-vessel plate sealing the vessels, so as to avoid, or substantially limit, evaporation and contamination of the samples.

The embodiments of the above described apparatus have been described in Example 1.

Another aspect of the invention relates to a rapid, high-throughput process of analyzing one or more fatty acids in a plurality of samples. The process comprises introducing a plurality of samples containing one or more fatty acids to individual vessels in a multi-vessel plate; mixing an esterification agent with each sample in the multi-vessel plate to produce esterified fatty acids; contacting the multi-vessel plate with a multi-vessel plate pre-heated to an esterification temperature of 50 to 300° C., separating the esterified fatty acids from each sample; and analyzing the esterified fatty acids from each sample by gas or liquid chromatography. Each vessel of the multi-vessel plate is sealed by a matching multi-cap mat.

This process can be performed in the apparatus described above. Thus, at least one of the introducing, mixing, contacting, separating, and analyzing steps is an automated step, carried out by an automated fluid handler and/or an automated multi-vessel plate handler described in the embodiments for the apparatus.

Any fatty acid known to one skilled in the art can be analyzed using the method, including saturated, unsaturated, and polyunsaturated fatty acids. Exemplary fatty acids to be analyzed include any fatty acid under the category of Omega-3 fatty acid, Omega-6 fatty acid, trans-isomeric unsaturated fatty acid, cis-isomeric monounsaturated fatty acid, saturated fatty acid, or combinations thereof. For instance, the method can be used to analyze fatty acid composition containing one or more of trans-palmitoleic, trans-oleic, trans-linoleic, cis-palmitoleic, cis-oleic, cis-eicosenoic, cis-nervonic, α-linolenic, eicosapentaenoic, docosapentaenoic, docosahexaenoic, linoleic, γ-linolenic, arachidonic, eicosadienoic, dihomo-γ-linolenic, docasatetraenoic, docosapentaenoic, myristic, palmitic, behenic, lignoceric, arachidic, or stearic acid.

The process may be used to analyze a fatty acid composition from any biological sample containing fatty acids or derivatives thereof. For instance, the biological sample can be a blood component such as whole blood, plasma, serum, red blood cells, platelets, white blood cells, cholesterol esters, triglycerides, free fatty acids, plasma phospholipids, or mixtures thereof.

The fatty acid to be analyzed may exist as various forms in the biological sample, such as triglycerides, diglycerides, monoglycerides, sterol esters, phosphatidyl ethanolamines, phosphatidyl cholines, free fatty acids, etc.

Thus, before analyzing the fatty acids or their derivatives in the biological sample, a single step of esterification can be used to convert these fatty acids or their derivatives into fatty acids esters. The esterifying agent can be any alcohol suitable for use in a typical esterification reaction to convert fatty acids or their derivatives into fatty acids esters. For instance, the esterifying agent can be a lower monovalent alcohol having 1 to 4 carbon atoms, such as methanol, ethanol, isopropanol, and butanol. Typical esterifying agent used is methanol, which can be use to convert any fatty acid to be analyzed to prepare a fatty acid methyl ester. Exemplary fatty acid esters to be analyzed contain one or more methyl esters of trans-palmitoleic, trans-oleic, trans-linoleic, cis-palmitoleic, cis-oleic, cis-eicosenoic, cis-nervonic, α-linolenic, eicosapentaenoic, docosapentaenoic, docosahexaenoic, linoleic, γ-linolenic, arachidonic, eicosadienoic, dihomo-γ-linolenic, docasatetraenoic, docosapentaenoic, myristic, palmitic, behenic, lignoceric, arachidic, or stearic acid.

An alkaline or an acidic catalyst can be used for esterification of fatty acids. An exemplary catalyst is BF₃. Additional esterification catalysts may include methanolic hydrogen chloride, methanolic sulfuric acid, and methanolic aluminum trichloride. The temperature for the esterification reaction typically ranges from about 60 to about 110° C. For instance, the temperature may range from 100 to 105° C. Temperatures outside of these ranges may also achieve esterification, but with less control over the time required to complete the reaction.

An exemplary method that includes a step of mixing of the esterification agent with each sample in the multi-vessel plate to produce esterified fatty acids involves adding the esterification agent into each sample in the multi-vessel plate, and vortexing the mixture in each vessel. The multi-vessel plate may then be contacted with a multi-vessel plate pre-heated to an esterification temperature, so that the reaction mixture in each sample vessel in the plate is simultaneously, evenly, and instantaneously (or nearly simultaneously, evenly, and instantaneously) brought to the desired temperature. During this time, each vessel of the multi-vessel plate may be sealed by a matching multi-cap mat. These steps can be carried out with an automated liquid handling device, automated homogenization (e.g., automated shaking, mixing, or vortexing), automated heating device, and/or a device enables simultaneously automated homogenization and heating, as described herein.

After the esterification step, the esterified fatty acid can be separated from each sample vessel for further analysis. The separating step may involve mixing an aqueous solvent with each esterified sample in the multi-vessel plate; simultaneously centrifuging the mixture in each vessel of the multi-vessel plate; and extracting the organic layer containing the esterified fatty acid from the centrifuged mixture in the multi-vessel plate. The separating step typically involves transferring the extracted sample components from the multi-vessel plate to a second multi-vessel plate for holding and measuring the transferred sample components. The second multi-vessel plate can be sealed immediately or soon thereafter with matching multi-vessel plate cap to avoid, or reduce, sample evaporation and contamination. The above steps can be carried out with an automated liquid handling device, as described in the embodiments for the apparatus.

For quantitative analysis of fatty acids in the sample, an internal standard can be added to each sample in the multi-vessel plate. The internal standard is used for calibration, for instance, by plotting the ratio of the fatty acid sample signal to the internal standard signal as a function of the analyte concentration present in the standards. Exemplary internal standards include those that are hydrophobic and have a molecular weight close to the total molecular weight of the fatty acids of interest. For instance, the internal standard can be any one of the fatty acids or derivatives that can be easily distinguishable from the tested fatty acids or derivatives from the sample. For example, an internal standard comprising the fatty acid C13:0, or the fatty acid C23:3n3 may be used. The internal standard can be a deuterated internal standard. When a deuterated internal standard is used, the deuterated internal standard can be a deuterated form of any one or more of the fatty acid to be analyzed.

The process thus can further involve a step of adding an internal standard to each sample in the multi-vessel plate. The internal standard can be dissolved in a solvent. Exemplary solvents include hexane. Suitable solvents also include acetone, acetonitrile, chloroform, ethylacetate, hexanes, isooctane, methanol, methylene chloride, petroleum ether, 2-propanol, tetrahydrofuran, toluene, and water. An internal standard can be added after the fatty acid samples are introduced to the multi-vessel plate, prior to the mixing step, prior to the contacting step, prior to the separating step or prior to the analyzing step. Typically, the internal standard is added immediately after the samples are introduced to the multi-vessel plate. The addition of an internal standard can be carried out with an automated liquid handling device, as described herein.

The process can further involve step of labeling the plurality of samples in the multi-vessel plate, and detecting the labeled samples for a sequential processing. The labeling step can be carried out by an automated bar-coding equipment described in the embodiments for the apparatus. The detecting step can be carried out by an automated bar code detector described in the embodiments for the apparatus.

The process can further involve analyzing the esterified fatty acids from each sample by gas or liquid chromatography. This analysis step can further include detecting the esterified fatty acids by a mass spectrometry, flame ionization detector or a thermal conductivity detector.

A detailed description of the analysis of fatty acid sample is shown in Example 2.

### EXAMPLES

The following examples are given as particular embodiments of the invention and to demonstrate the practice and advantages thereof. It is to be understood that the examples are given by way of illustration and are not intended to limit the specification or the claims that follow in any manner.

### Example 1

### An Exemplary Apparatus

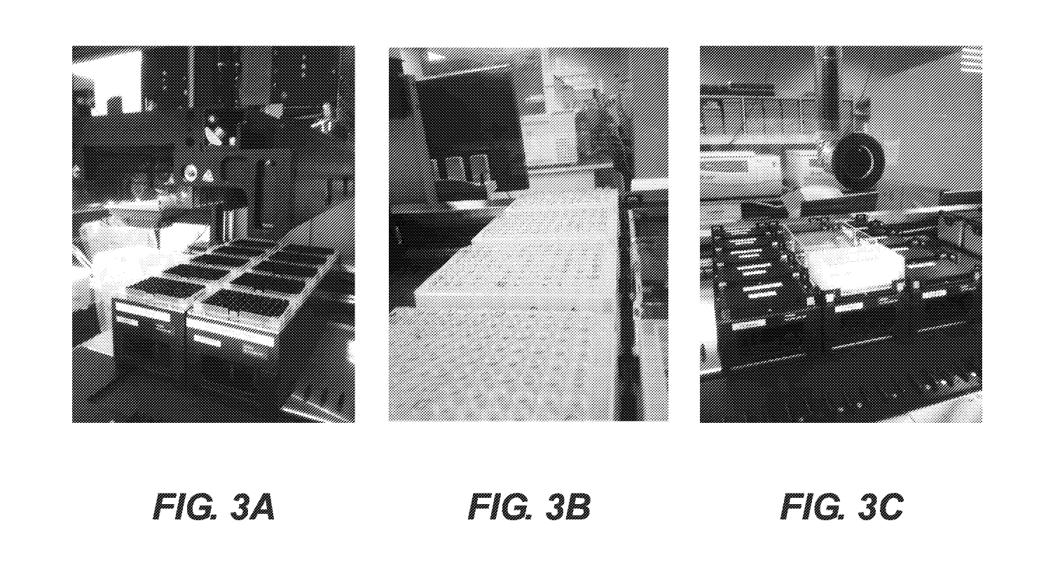

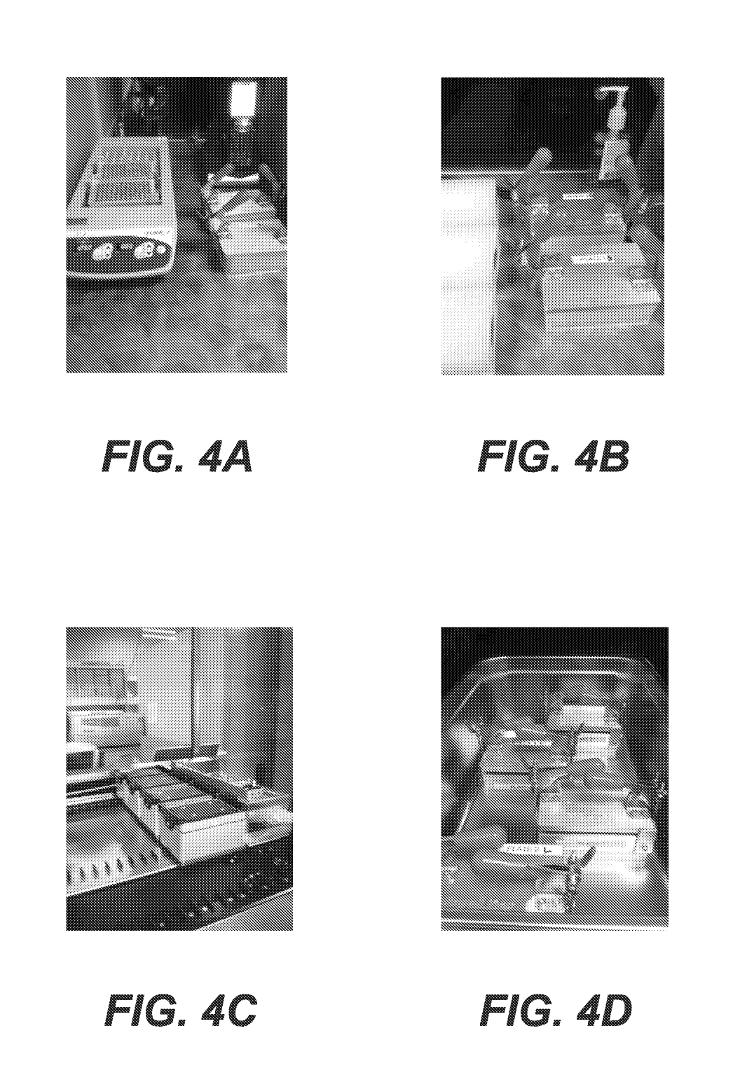

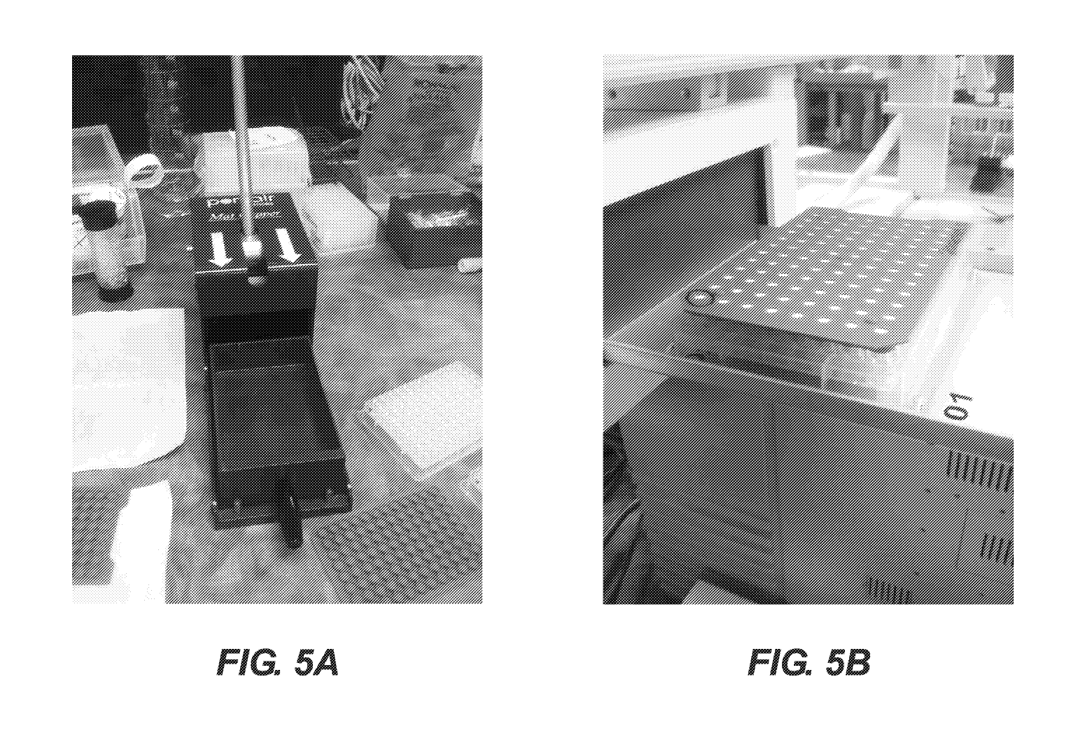

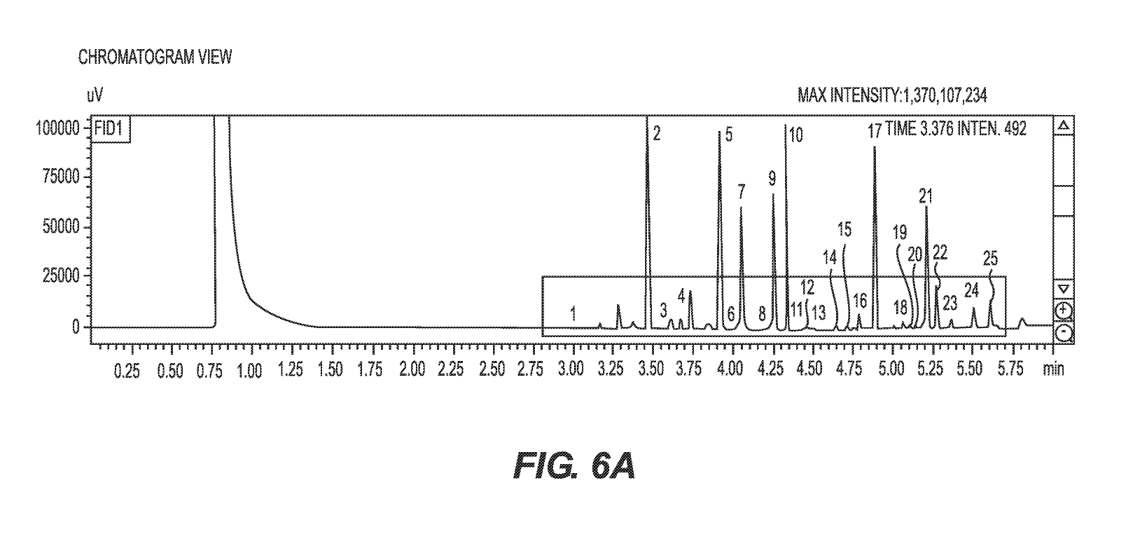

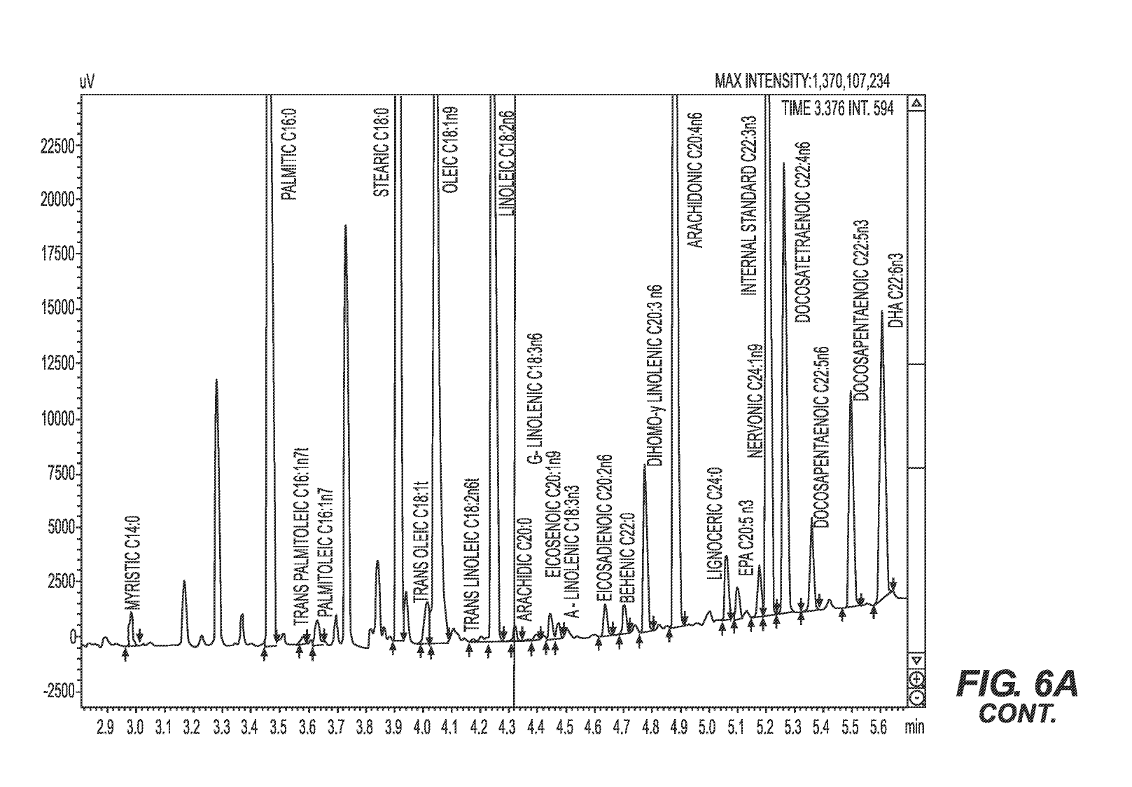

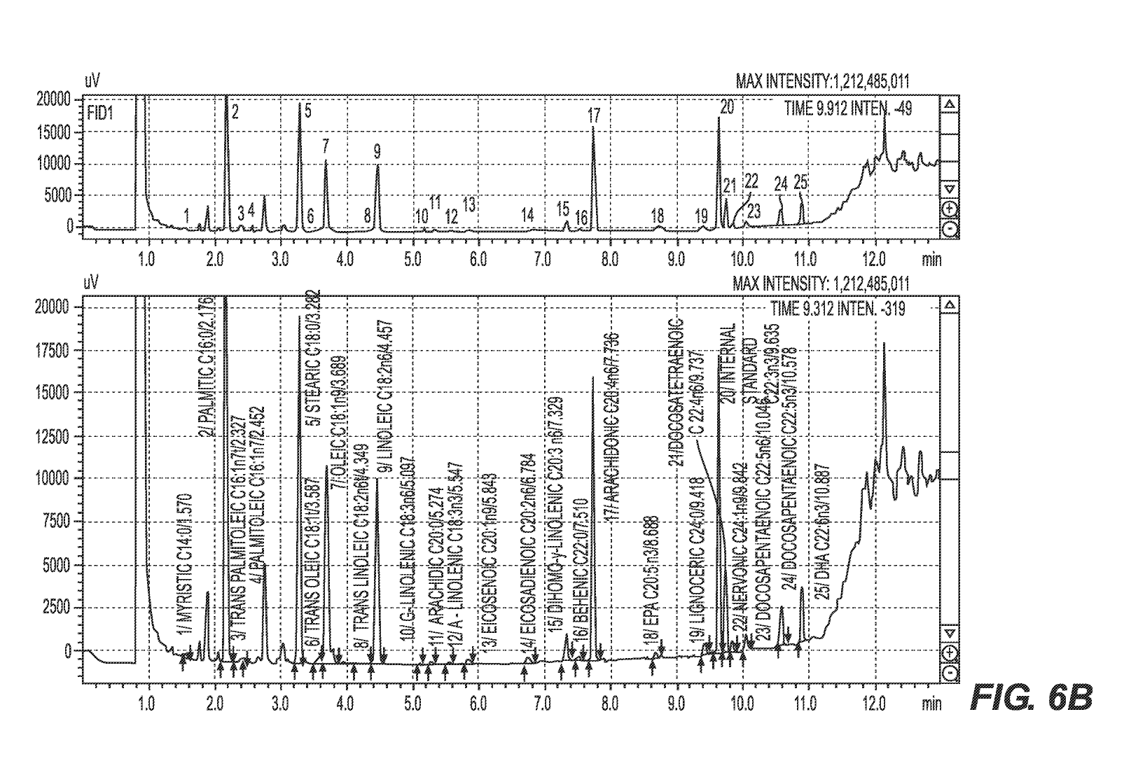

An apparatus for high-throughput esterifying and analyzing fatty acids include the following exemplary elements (see FIGS. 3-5):

Hamilton Microlab Star liquid handling system with 96 head pipetter, tip cutter, heater shakers, glass reagent troughs;

CapMat Vise (MicroLiter Analytical/07-0000-C) to seal the 96 vial cap mat on top of the 96 vial plate;

MicroLiter aluminum block with clamps (MicroLiter Analytical/07-HTGB-1000HP) for sealing the aluminum block and allowing for constant high temperature and high pressure heating of 1.2 mL glass tubes containing methanol without evaporation or explosion;

MicroLiter 150 μL glass inserts in 96 well plate+mat (VWR 89212-428) for use on the GCFID autosampler with no interference leaching from the inserts or mat;

MicroLiter 1.2 mL glass inserts in 96 well plate+mat (VWR 89212-426) for use in the aluminum block with no interference leaching from the inserts or mat;

Conductive 300 μL tips with 5 mm cut (Hamilton/235902) so that the bore of the tip is wide enough to accurately pipette samples (e.g., packed red blood cells);

Conductive 50 μL tips (Hamilton/235966) or equivalent;

Slim tips 300 μL (Hamilton/235806) having specific slim tips to accurately pipette the solvent (e.g., hexane and/or methanol) and to prevent dripping;

Barcode scanner;

Aluminum block heater shakers;

Chilling water bath to cool the aluminum blocks quickly.

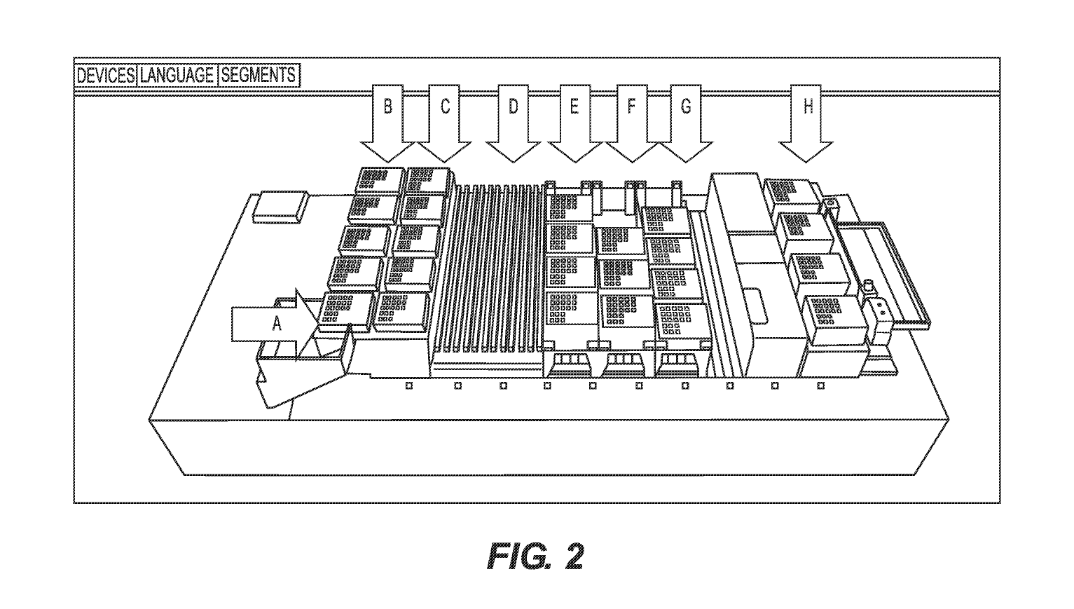

FIG. 2 shows exemplary elements for an apparatus for high-throughput esterifying and analyzing fatty acids:

A—front 2 trays are for 300 μL filtered tips (barcodes to right)

B—back 4 trays in first row are for 50 uL filtered tips (barcodes to right)

C—back 4 trays in second row are for 300 uL filtered tips, cut or slim tips (barcodes to right)

D—sample racks

E—1.2 mL glass inserts in 96 well plate—plate 1 in front position, plate 4 at very back (barcode to right)

F—reagent troughs—RO water in front trough, Hexane/IS in 2^{nd} trough, BF3 in 3^{rd} trough

G—150 μL glass inserts in 96 well plate

H—heater shakers

### See FIGS. 2-5.

### Example 2

### Methods for Automated, High-Throughput Analysis of Fatty Acids

The following exemplary procedures have been programmed in Hamilton Microlab STAR system to illustrate the fatty acid sample esterifications, separations, and detections using the automated apparatus including the multi-vessel plates with matching multi-cap mats and matching multi-vessel plate holder, automated liquid handling devices, automated multi-vessel plate heating/homogenization unit, automated multi-vessel plate separating unit, automated multi-vessel plate measuring unit, automated labeling equipment and a label detector, and the data processing and control software, as described in the above embodiments.

Exemplary procedures for a high-throughput analysis of fatty acids are shown as below.

Hamilton scanned all sample barcodes and plate barcodes in order. Hamilton saved these barcode numbers as load lists, which allows continuous tracking of specimens to assure no sample mix-ups.

To each well (a 1.2 mL glass vial) in a 96 deep well plate, Hamilton pipetted 25 μL of red blood cell (RBC), and 250 μL of BF₃ in methanol (e.g., 14% methanol), followed by 250 μL of hexane/internal standard (IS).

The 96 deep well plate was removed from Hamilton and the matching multi-vessel mat was placed on top of the plate. The plate with the matching multi-vessel mat was placed in the CapMat Vise and sealed.

The 96 deep well plate was then placed in a multi-vessel vortexer and vortexed for approximately 1-2 minutes.

The program in Hamilton was set to prompt “Secure samples in the pre-heated aluminum blocks. Move blocks to Heater Shakers,” when the heater shakers are heated to the correct temperature. In this example, the digital heat block was set to approximately 105° C. (external thermometer read approximately 100° C.). Once the prompt appeared on the Hamilton, showing that the heater shakers reached corrected temperature, the aluminum block was removed from the digital heat block. The cap mat was quickly moved with the vials attached into the pre-heated block and the cap mat was then clamped down. The plate was placed on the appropriate position in area H in FIG. 2. Hamilton then locked the plate.

After 10 minutes, the aluminum block carrier was moved to the approved cooling device. The cap mat was removed from the plate. The plate was placed back into the original position in area H on Hamilton.

Hamilton added 250 μL of reverse osmosis water or HPLC grade water into each well in the plate. The 96 deep well plate was then taken offline and vortexed for approximately 1-2 minutes. Thereafter, the plate was spun in centrifuge at 3000-3500 rpm for 10 minutes.

The above-processed 96 deep well (1.2 mL glass vial) plate was returned to source position on the Hamilton.

A 96 well (150 μL glass vial) plate was placed onto Hamilton in correct position in area G. Hamilton transferred 120 μL of the organic layer from each well of the 96 deep well (1.2 mL glass vial) plate to the corresponding well in the 96 well (150 μL glass vial) plate for subsequent gas chromatography (GC).

The 96 well (150 μL glass vial) plate was placed in the CapMat Vise and the cap mat was then clamped down. The plate was removed, turned, and clamped down again in an effort to ensure a complete fit and seal. The samples in the 96 well (150 μL glass vial) plate was injected on a Shimadzu 2010 gas chromatography flame ionization detection (GCFID) with a Reztek Rt-2560 column.

### Example 3

### Validation of Automated, High-Throughput Process for Analyzing Fatty Acids by GC-FID (Gas Chromatography-Flame Ionization Detection)

Fatty acid sample esterifications, separations, and detections using the automated apparatus were carried out according to the exemplified procedures described in Examples 1 and 2. The experiments in this example demonstrate the validation set-up for the automated assay for the determination of fatty acids in Red Blood Cells (RBC) by GC-FID.

### Instrumentation and Parameters

The following instruments, equipments, and parameters were employed in the validation method.

Hamilton Microlab Star “Hasselhoff”;

Shimadzu GC 2010: “Sabertooth”;

Shimadzu GC 2010 Plus: “Lady Liberty,” “A1 The Octopus,” “Tommy Hawk,” “Youppi,” “Carlton,” “Blade”;

rocking platform;

digital heat block;

multi-vessel plate vortexer;

multi-vessel centrifuge;

balance;

repeating pipette;

zippette bottle-top dispenser;

screw-cap test tube, 2 ml (Kimble/60810-1528);

teflon-lined screw-cap (CapMat Vise) (Qorpak/CAP-00545);

96-well plate (Greiner—VWR #780261);

GC vials with inserts (VWR HP-9301-1388);

Crimp top caps for GC vials (VWR HP-5061-3370)

Parameters for Shimadzu GC 2010 Plus:

1. Injection Port SPL 1
i. Injection Volume: 2 μL
ii. Injection Mode: Split
iii. Temperature: 250° C.
iv. Carrier Gas: H₂
v. Flow Control Mode: Linear Velocity
vi. Pulsed Injection Pressure: 160 kPa for 0.1 minute
vii. Inlet Pressure after 0.1 min 130.7 kPa
viii. Total Flow: 29.6 mL/min
ix. Column Flow: 2.42 mL/min
x. Linear Velocity: 70 cm/sec
xi. Purge Flow: 3.0 mL/min
xii. Split Ratio: 10.0
xiii. High Pressure Injection: On
xiv. Carrier Gas Saver: Off
xv. Splitter Hold: Off

2. Column Oven
i. Initial Temperature: 165.0° C.
ii. Equilibration Time: 0.5 min
iii. Temperature Program:
   1. Total Program Time: 12.98 min

| a. | Rate (° C./min) | | Temperature (° C.) | Hold Time (min) |
|---|---|---|---|---|
| | i. | — | 165.0 | 5.00 |
| | ii. | 7.0 | 208.0 | 0.00 |
| | iii. | 50.0 | 250.0 | 1.00 |

3. Column Information
i. Column Name: HP-88
ii. Film Thickness: 0.20 μm
iii. Column Length: 30.0 m
iv. Inner Diameter: 0.25 mm
v. Column Max Temp: 250/260° C.

4. Detector 1 FID 1
i. Temperature: 260° C.
ii. Sampling Rate 40 msec
iii. Makeup Gas: He
iv. Makeup Flow: 30.0 mL/min
v. H2 Flow: 40.0 mL/min
vi. Air Flow: 400.0 mL/min

### Materials

Reagents used in the method: BF₃ with 14% methanol (Sigma-Aldrich B1252); n-hexane (VWR Alfa Aesar 43263); acetone (VWR B&J 010-4); and Agilent Column HP88.

Quality control (QC) material used in the method: Supelco® 37 Component FAME (Fatty Acid Methyl Esters) Mix (Sigma-Aldrich 47885-U); and pooled and aliquotted whole blood containing low and high levels of omega-3 fatty acid.

Standard materials used in the method: GLC-A (a gas-liquid chromatography standard, prepared as shown in the “III. Stock Standard Preparation” below); and Omega Quant Standard3 (prepared as shown in the “IV. Standard Curve Preparation” below).

### Method

### I. Standard Preparation.

Top stock Standard (1000 μg/mL). 10 mg of each standard was measured into individual 20 mL scintillation vials. 10 mL of ethanol was added to each standard, and then each standard was sonicated for approximately 5-10 minutes. All stock standards were stored at −20° C.

Spiking standard (10 μg/mL). 100 μL of each standard was pipetted into a 10 mL volumetric flask and diluted with ethanol to 10 mL, and then was sonicated for approximately 5-10 minutes. The standards were transferred to 20 mL scintillation vials and store at −20° C.

### II. Internal Standard (IS) Preparation.

C22:3 n-3 FAME Internal Standard stock solution (2.5 mg/mL) was made according to the following steps: 25 mg of C22:3 n-3 FAME (thawed at room temperature) was added in a 10 mL volumetric flask that was rinsed with n-hexane (×3), and diluted with n-hexane to 10 mL. 1.2 mL of stock solution was transferred into a 2 mL vial, topped with Argon, and stored at −80° C. until needed.

C22:3 n-3 FAME Internal Standard working solution (12.5 μg/mL) was made by diluting the stock solution. 500 μL of C22:3 n-3 stock solution was added to 100 mL volumetric flask and diluted to 100 mL with n-hexane. The resulting solution was mixed and transferred to an amber jar with Teflon/PTFE (polytetrafluoroethylene)-lined cap, and store at 2-8° C.

### III. Stock Standard Preparation

GLC-A Standard Stock Solution (2.5 mg/mL) was prepared according to the following steps. 25 mg of GLC-A (thawed at room temperature) was added in a 10 mL volumetric flask that was rinsed with n-hexane (×3), and diluted with n-hexane to 10 mL. 0.5 mL of stock solution was transferred into a 2 mL vial, topped with Argon, and store at −80° C. until needed.

### IV. Standard Curve Preparation

GLC-A Standard Curve Levels (250, 100 and 10 μg/mL) were prepared according to the following steps.

Level 3 (OQStandard3-3)—250 μg/mL. 1.78 mL of n-hexane, 0.2 mL of GLC standard stock solution and 0.02 mL of C22:3 n-3 Internal Standard (IS) stock solution were added into a 2 mL vial.

Level 3 prepared without IS. 1.78 mL of n-hexane and 0.2 mL of GLC stock solution were added into a 2 mL vial.

Level 2 (OQStandard3-2)—100 μg/mL. 1.18 mL of n-hexane, 0.8 mL of Level 3 without IS, and 0.02 mL of C22:3 n-3 IS stock solution were added into a 2 mL vial.

Level 1 (OQStandard3-1)—10 μg/mL solution. 1.18 mL of n-hexane, 0.08 mL of Level 3 without IS, and 0.02 mL of C22:3 n-3 IS stock solution were added into a 2 mL vial

### V. Sample Preparation:

1. The tubes containing whole blood were placed on a rocking platform for approximately 10 minutes to ensure adequate mixing of the specimen;
2. Hamilton Star was used to aliquot 500 μL of specimen into 96-well plate;
3. The plate was centrifuged for 10 minutes at 3500 rpm;
4. Hamilton Star was used to pipette 50 μL of packed cells into screw-top tubes;
5. A manual repeat dispenser was used to pipette 500 μL of BF₃ w/14% methanol into each screw-top tube;
6. A manual repeat dispenser was used pipette 500 μL of hexane to each screw-top tube;
7. The tubes were then capped and vortexed rapidly for approximately 30 seconds, to ensure complete mixing of the specimen and solvent;
8. The tubes were then incubated at approximately 100° C. for 10 minutes.
9. The tubes were removed from water bath and allowed to cool for approximately 10 minutes.
10. 500 μL of HPLC-grade water was added to each tube with a manual repeat dispenser and vortexed for 30 seconds.
11. The tubes were centrifuged at 3500 rpm for 10 minutes.
12. Approximately 50 μL of the organic layer was transferred to a GC vial with insert using the Hamilton Star.
13. Samples were injected under standard GC method.

### Example 4

### Validation of Automated, High-Throughput Process for Analyzing Fatty Acids by GC-FID via Comparison with Manual Process

Validation is a useful guidepost when developing and implementing a novel bioanalytical method. In this example, the automated, high-throughput process was compared with a manual process for method validation.

Validations of the automated, high-throughput process for fatty acid analysis have been performed, complying with standard operating procedures of Food and Drug Administration (see, U.S. Department of Health and Human Services, Food and Drug Administration, “Guidance for Industry Bioanalytical Method Valication,” (May 2001)).

### Validation Scope:

Validation studies were performed on EDTA (ethylenediaminetetraacetic acid)—packed red blood cells (RBC). Calibrators, quality control materials, and patient samples were assayed to determine the following analytical characteristics of the clinical assay:

Accuracy

Reference Range Verification

Stability

Intra Assay Precision-3 samples assayed 20 times each on a single run

Inter Assay Precision-3 samples assayed in singlicate over 20 runs on minimum of five days

Spike and Recovery

Analytical Sensitivity

Analytical Measurement Range (AMR) Linearity

Carryover

Limit of Detection

The following analytes and groups of analytes were evaluated for each of the studies:

### Analytes

Linoleic acid C18:2n-6

Arachidonic Acid (AA) C20:4n-6

Eicosapentainoic Acid (EPA) C20:5n-3

Docosahexaenoic Acid (DHA) C22:6n-3

### Fatty Acid Families

Omega-3 polyunsaturated fatty acids (PUFA)

Omega-6 PUFA

Monounsaturated fatty acids

Saturated fatty acids

### Fatty Acid Indices

HS-Omega-3 Index®

Trans-Fat Index

### Accuracy Verification

Accuracy verification of a method describes the closeness of mean test results obtained by the method to the true values (concentration) of the analyte. Accuracy verifications were performed on a minimum of 120 different specimens (approximately 60 males and 60 females that were all 18 years of age or older) that varied in concentrations.

The specimen were spun down and aliquotted for testing. The results for mean % and mean absolute difference for measured analytes and fatty acid families are shown in Table 1. The results in Table 1 demonstrate the mean % or mean absolute difference for all measured analytes are within acceptance criteria.

TABLE 1
Mean % and mean absolute differences of fatty acids and
fatty acid families in RBCs for 120 different specimen
measured by automated, high-throughput process.

| | Mean % Dif | Mean Abs Dif |
|---|---|---|
| Individual | | |
| Myristic C14:0 | NA | 0.04 |
| Palmitic C16:0 | −6.12 | NA |
| Trans Palmitoleic C16:1n7t | NA | 0.09 |
| Palmitoleic C16:1n7 | NA | −0.22 |
| Stearic C18:0 | −7.25 | NA |
| Trans Oleic C18:1t | NA | 0.43 |
| Oleic C18:1n9 | 7.61 | NA |
| Trans Linoleic C18:2n6t | NA | 0.10 |
| Linoleic C18:2n6 | 12.82 | −1.47 |
| G-Linolenic C18:3n6 | NA | 0.07 |
| Arachidic C20:0 | NA | −0.01 |
| A-Linolenic C18:3n3 | NA | −0.02 |
| Eicosenoic C20:1n9 | NA | −0.13 |
| Eicosadienoic C20:2n6 | NA | −0.02 |
| Dihomo-y-linolenic C20:3n6 | NA | −0.05 |
| Behenic C22:0 | NA | −0.15 |
| Arachidonic C20:4n6 | 2.52 | −0.08 |
| EPA C20:5n3 | NA | 0.03 |
| Lignoceric C24:0 | NA | −0.38 |
| Docasatetraenoic C22:4n6 | NA | 0.52 |
| Nervonic C24:1n9 | NA | −0.32 |
| Docosapentaenoic C22:5n6 | NA | 0.02 |
| Docosapentaenoic C22:5n3 | NA | 0.18 |
| DHA C22:6n3 | NA | −0.01 |
| Family | | |
| Omega 3 | −4.31 | 0.16 |
| Omega 6 | 12.64 | −1.45 |
| HDL Cis-Monounsaturated F | 12.05 | 0.48 |
| HDL Saturated FA | −5.34 | NA |
| HDL Trans FA | NA | 0.62 |
| HDL Omega 3FA index | 0.30 | 0.03 |

Acceptance criteria:
mean % difference: +/−20%, or mean absolute difference: +/−2 for those analytes that are found in amounts below 10% of the total composition of the RBC. “NA” was reported where the value was deceptively unrelated to the comparison. For example, in cases where the absolute values are low, the compared values would generate a deceptively large number. Thus, the percent differences for these values were not reported.

### Method Comparison

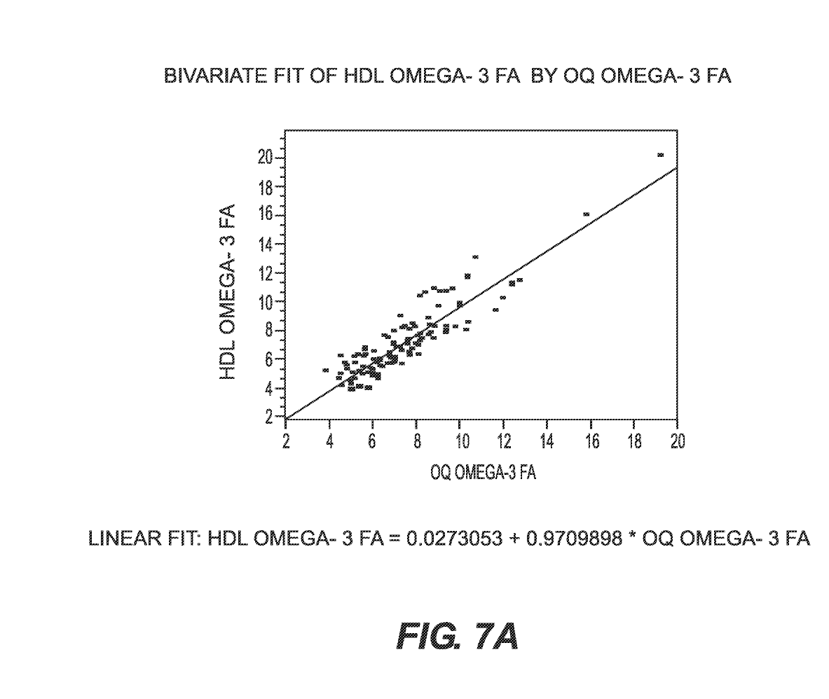

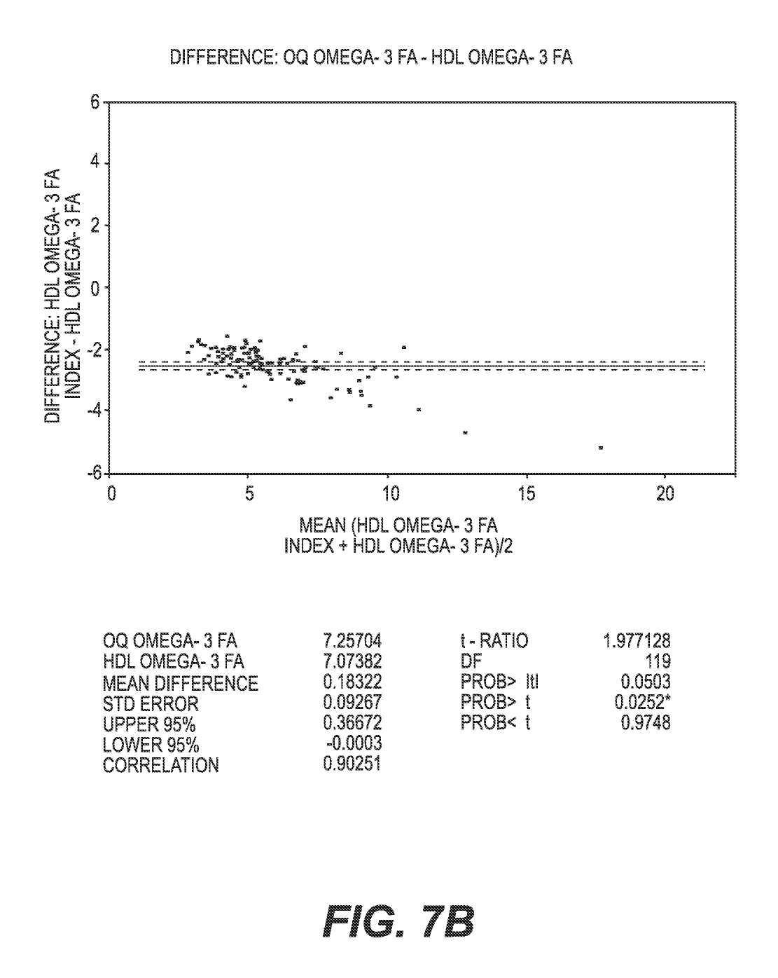

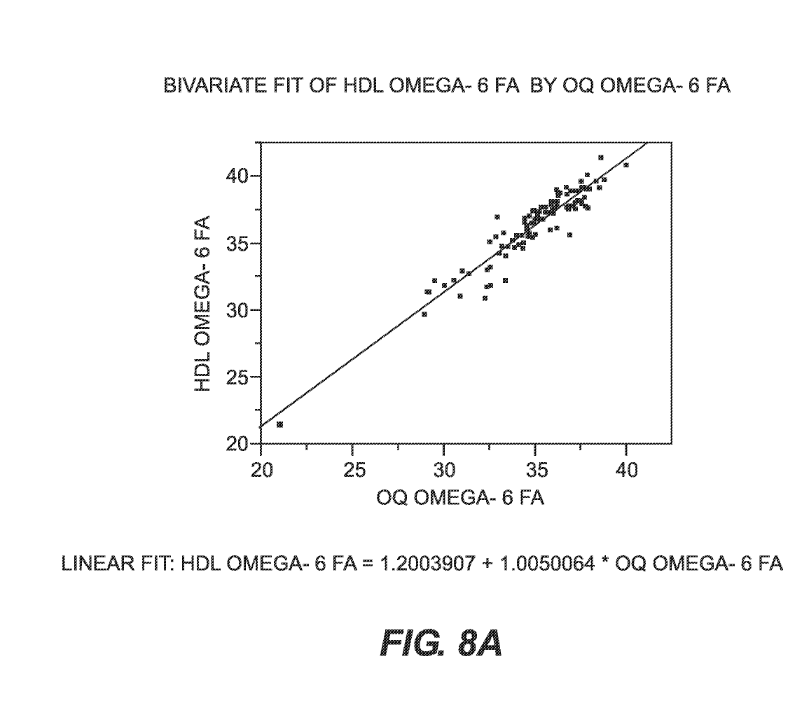

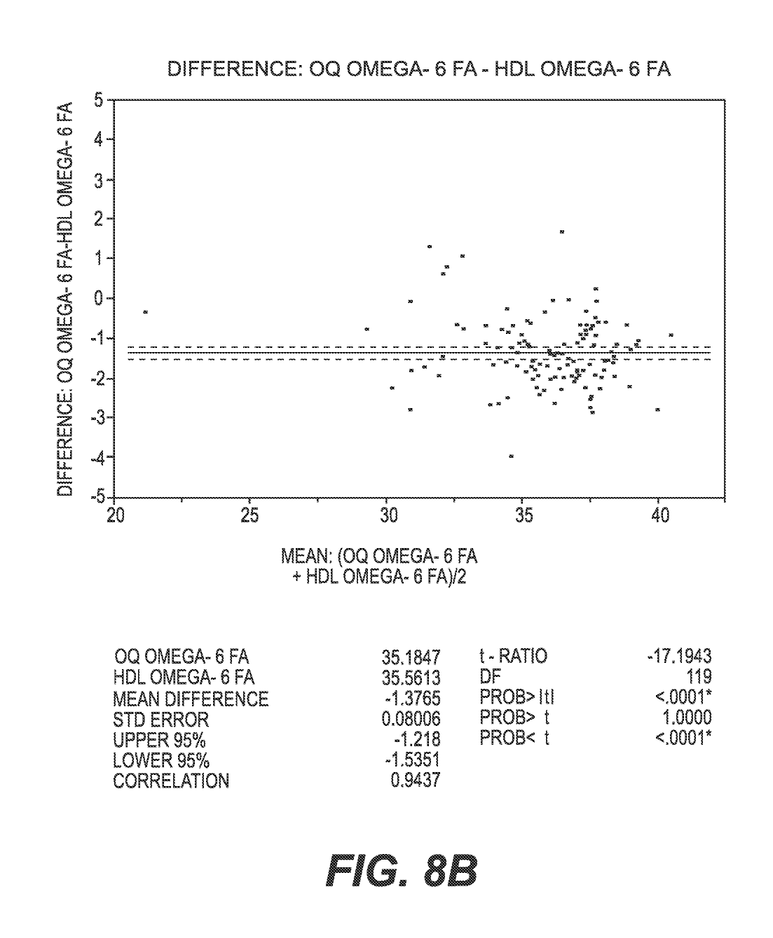

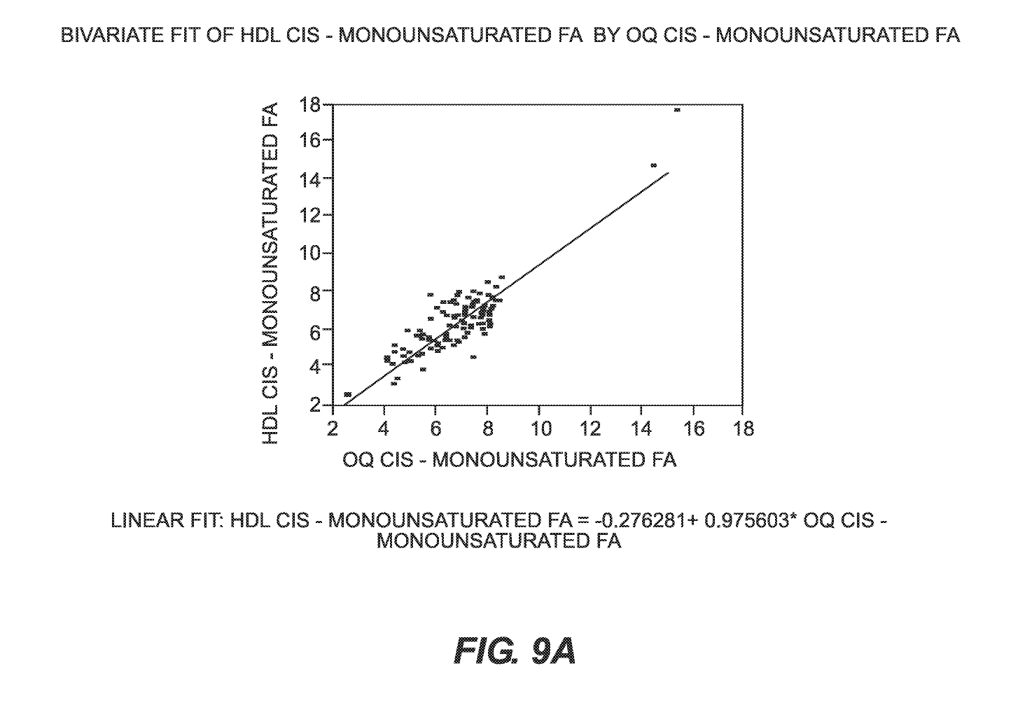

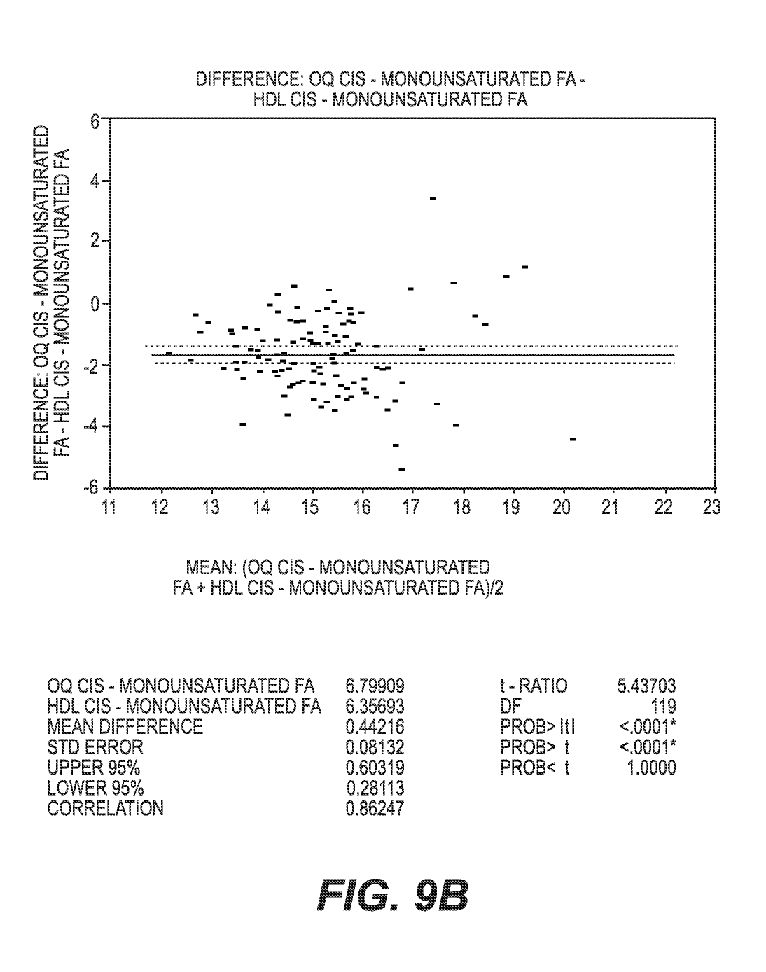

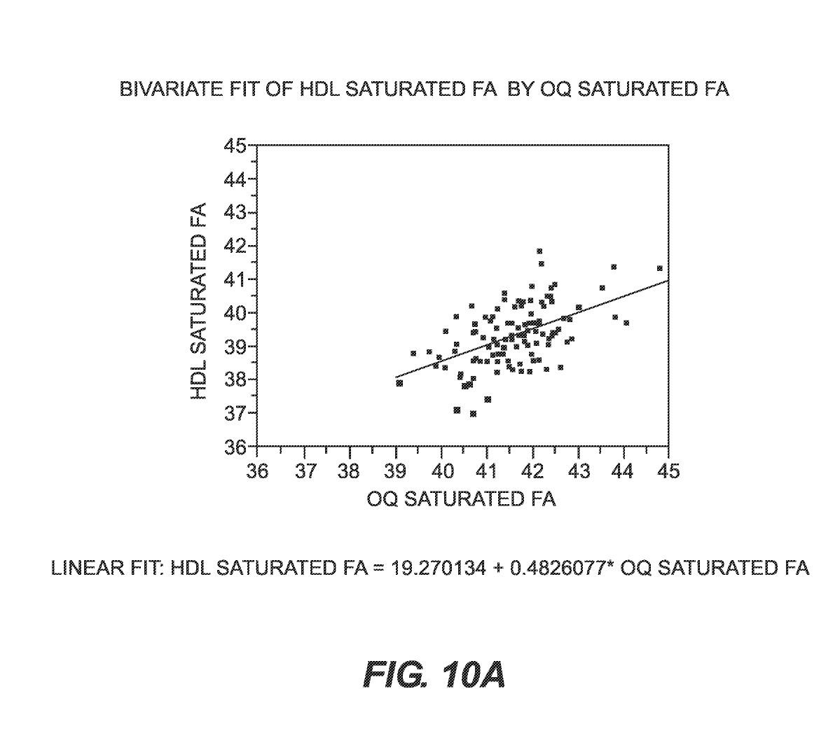

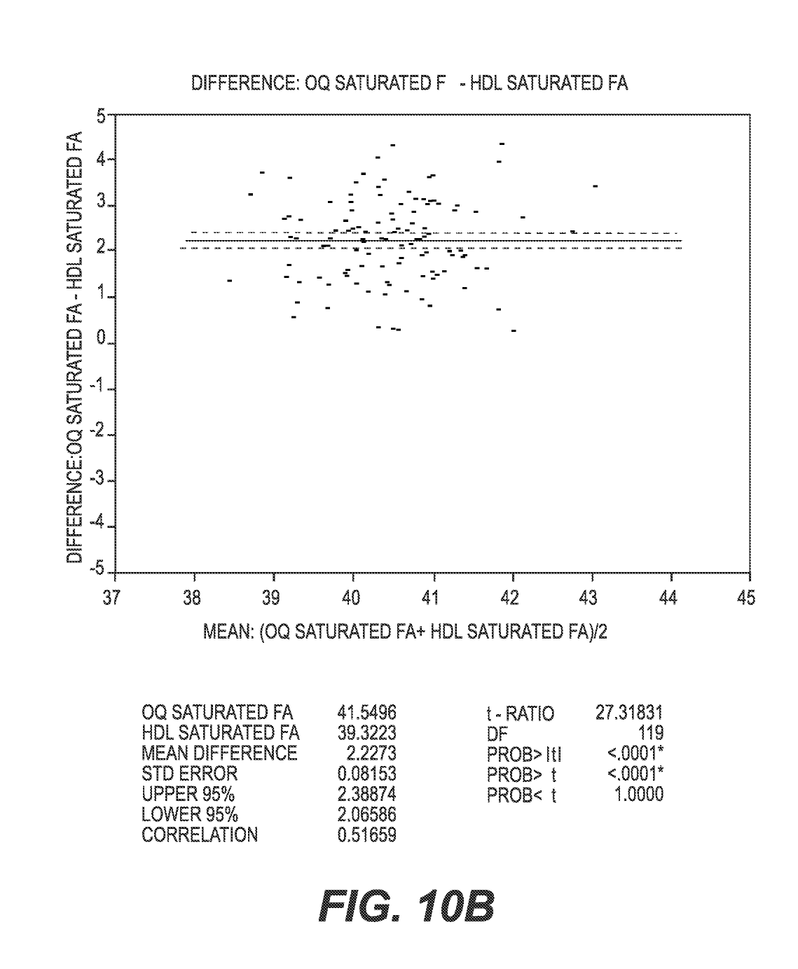

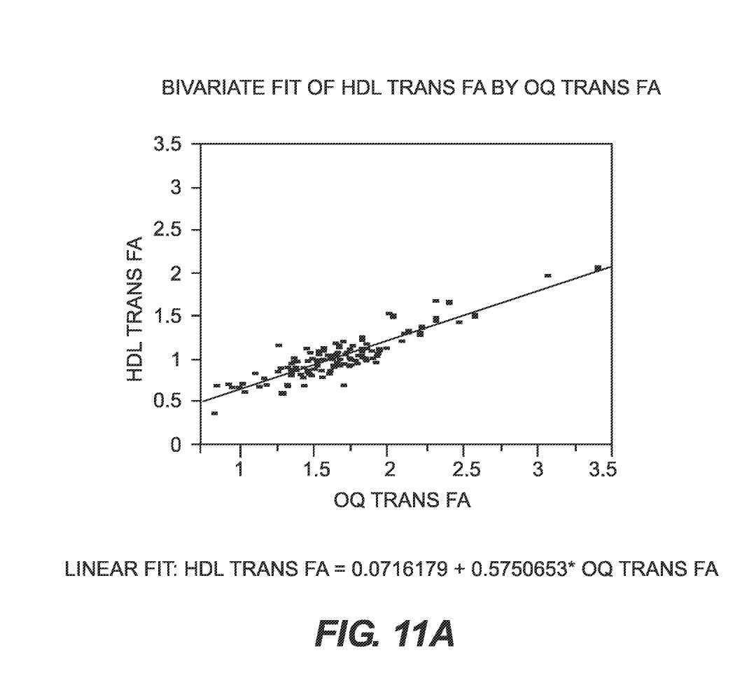

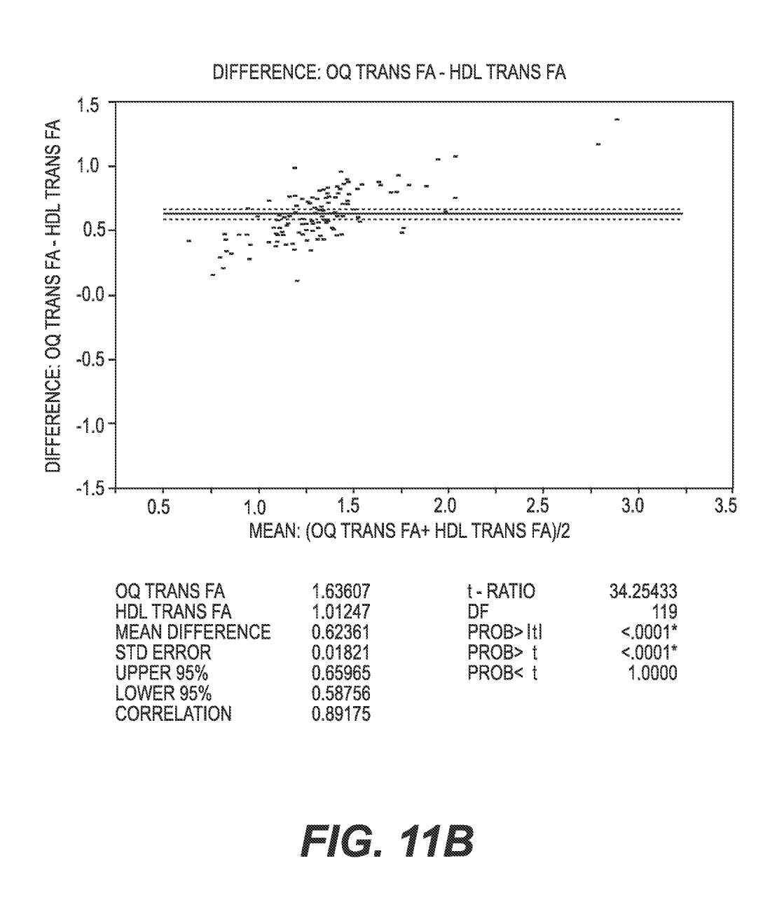

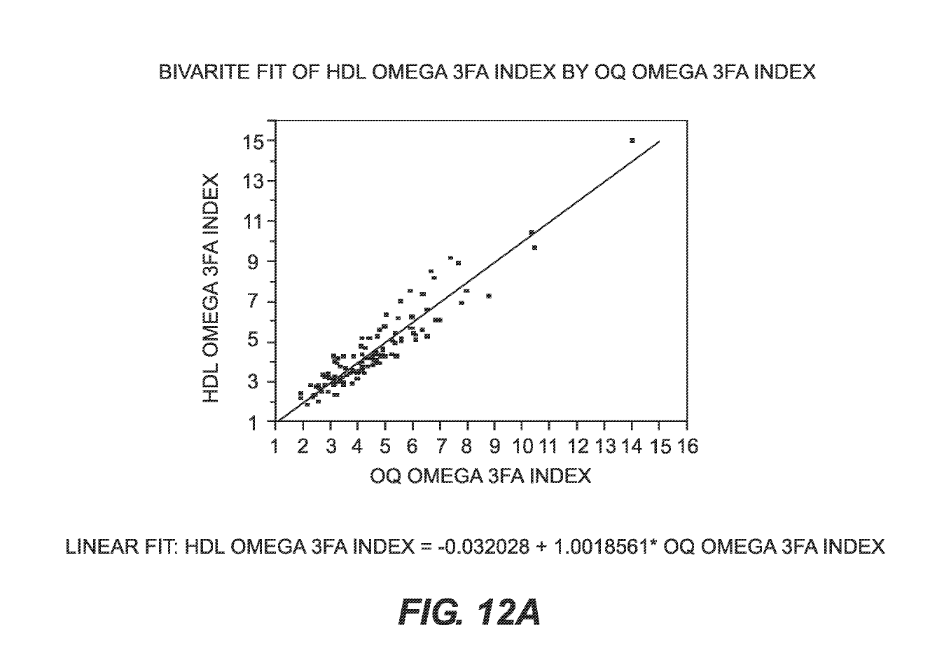

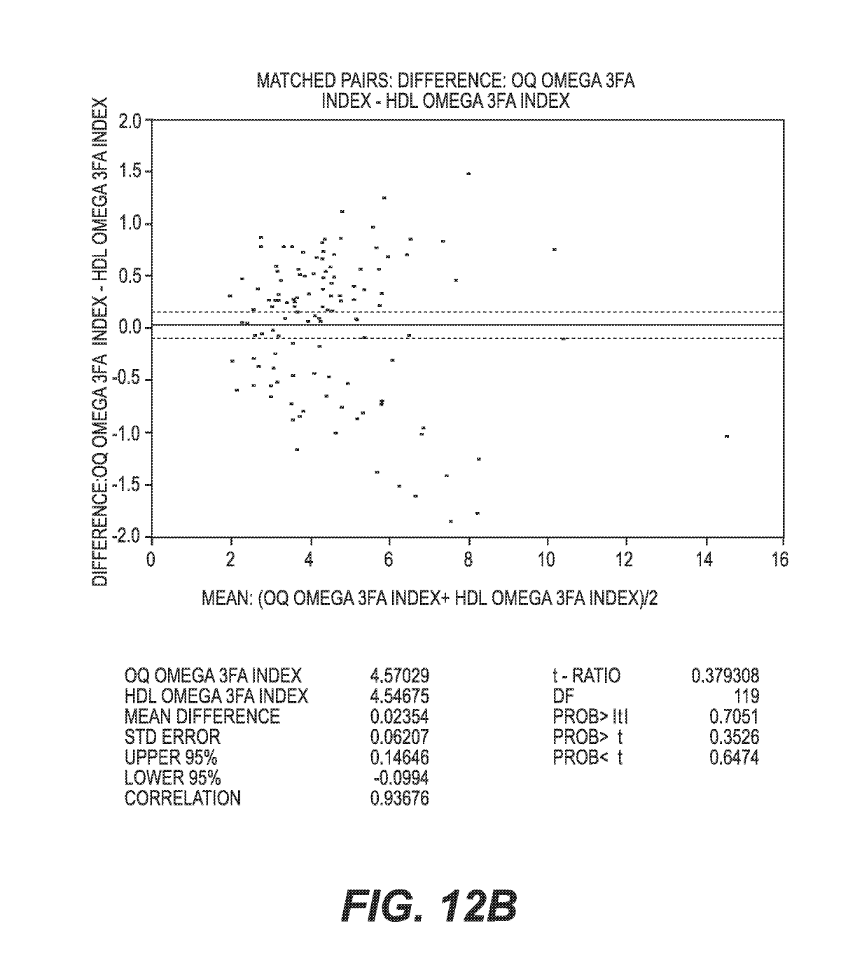

The analyses of the above analytes and fatty acid families by the automated, high-throughput process were compared to the Reference Method—Omega Quant (OQ) EDTA RBC analysis of the same specimens. The comparison results were analyzed by linear regression and Bland Altman plots. The results of comparing omega-3 fatty acid, omega-6 fatty acid, cis-monounsaturated fatty acid, saturated fatty acid, trans fatty acid, and omega-3 fatty acid index by automated high-throughput process and by manual process are shown in FIGS. 7-12. The automated, high-throughput method is exemplified as “HDL” in the graph and the manual method is exemplified as “OQ” in the graph. In FIGS. 7-9 and 12, all slopes, intercept and correlations were within acceptance criteria. In FIG. 10, the % difference between the OQ saturated FA and HDL saturated FA was −5% which was within acceptance criteria. In FIG. 11, the correlation and intercept were acceptable and the average absolute difference between the OQ trans FA and HDL trans FA was less than 2, which was within acceptance criteria. The analyses of figures showed that the automated, high-throughput process was achieving the same or similar results as the manual process. This cross-validation with an established manual process confirmed the accuracy of the automated, high-throughput process as a bioanalytical method.

### REFERENCE RANGE

As the accuracy of the above analytes and fatty acid families measurements have passed acceptance criteria, the corresponding measurement results were used to establish reference range for the automated, high-throughput process. In particular, reference ranges of the fatty acids in omega-6, cis-monounsaturated and trans fatty acid families were the established by using the 120 comparison results from the automated, high-throughput process. The reference ranges of the omega-3 index from the automated, high-throughput process can be the same ranges as those from the manual Omega Quant process. The distribution by gender showed no difference between genders for any of the fatty acid families.

The reference ranges of fatty acids and fatty acid families in RBCs determined from 120 different specimen by automated, high-throughput process were shown in Table 2, compared to the Framingham Cohort ranges. The automated, high-throughput method is exemplified as “HDL” in the table. The reference ranges shown in the table were +/−3SD of the mean values obtained by using the automated, high-throughput process. The omega-3 index reference range from the automated, high-throughput process, not shown in the table, includes high risk (<4%), intermediate (4-8%), and low risk (>8%).

TABLE 2
Reference ranges of fatty acids and fatty acid families in RBCs
determined from 120 different specimen by automated, high-throughput
process compared to the Framingham Cohort ranges.

| | Reference Ranges | | |
|---|---|---|---|
| | Framingham | | |
| | N = 3215 | HDL N = 120 | |
| | Framingham 3 | Range | Range |
| | SD Ranges (%) | Low (%) | High (%) |
| Myristic C14:0 | 0.1-0.8 | <0.1 | 0.67 |
| Palmitic C16:0 | 17.36-25.22 | 17.09 | 24.95 |
| Trans Palmitoleic C16:1n7t | 0.03-0.31 | <0.1 | 0.10 |
| Palmitoleic C16:1n7 | −0.22-0.94 | <0.1 | 1.66 |
| Stearic C18:0 | 15.12-21 | 13.13 | 20.78 |
| Trans Oleic C18:1t | −0.05-3.44 | <0.1 | 1.31 |
| Oleic C18:1n9 | 9.6-18.24 | 10.77 | 18.49 |
| Trans Linoleic C18:2n6t | 0.0-0.53 | 0.11 | 0.47 |
| Linoleic C18:2n6 | 6.04-16.19 | 4.70 | 21.31 |
| G-Linolenic C18:3n6 | −0.21-0.37 | <0.1 | 0.24 |
| Arachidic C20:0 | 0.1-0.3 | <0.1 | 0.27 |
| A-Linolenic C18:3n3 | −0.12-0.5 | <0.1 | 0.41 |
| Eicosenoic C20:1n9 | −0.06-0.61 | 0.17 | 0.52 |
| Eicosadienoic C20:2n6 | 0.11-0.44 | 0.17 | 0.48 |
| Dihomo-y-linolenic C20:3n6 | 0.45-2.76 | 0.60 | 3.19 |
| Behenic C22:0 | 0.1-0.2 | <0.1 | 0.44 |
| Arachidonic C20:4n6 | 11.95-21.63 | 10.58 | 23.32 |
| EPA C20:5n3 | −0.61-2.05 | <0.1 | 2.51 |
| Lignoceric C24:0 | −0.06-0.95 | 0.16 | 1.04 |
| Docasatetraenoic C22:4n6 | 1.31-6.28 | 0.72 | 6.53 |
| Nervonic C24:1n9 | −0.05-0.89 | <0.1 | 0.98 |
| Docosapentaenoic C22:5n6 | 0.1-1.23 | <0.1 | 1.34 |
| Docosapentaenoic C22:5n3 | 1.38-4.08 | 0.60 | 4.12 |
| DHA C22:6n3 | 0.76-8.89 | <0.1 | 8.41 |
| Family | | | |
| Omega 3 Total | 2.6-14.3 | <0.1 | 14.13 |
| Omega 6 Total | 25-36 | 28.61 | 44.51 |
| Cis-monounsaturated Total | 10.4-19.5 | 11.58 | 20.48 |
| Saturated Total | 36.3-43.3 | 36.69 | 41.95 |
| Trans Index | 0.1-3.9 | 0.26 | 1.76 |

### Instrument Cross Check and Column Reproducibility

Instrument cross check and column reproducibility studies were performed. All the GC instruments and columns used in the automated, high-throughput process were evaluated and cross-checked. The results verified excellent concordance and reproducibility between different GC instruments and the columns used in the automated, high-throughput process.

### Sample Stability and Stock Standard Stability

Sample stability and stock standard stability were assessed. The results indicate that all families and index families of fatty acids were stable when being frozen (−80° C.) and refrigerated for at least 14 days; the extracted samples were stable inside the auto sampler for at least 24 hours and the extracted samples were stable when left to sit on the bench for at least 4 hours. Moreover, stock standard used in the automated, high-throughput process was stable for at least 17 days. Accordingly, both sample stability and stock standard stability in the automated, high-throughput process were acceptable.

### Within-Run (Intra-Assay) Precision

Twenty replicates from each of at least three patient sample pools were measured in a single run. Although this process could be broken up over several runs, twenty replicates of each patient sample pool were at least measured within the same run. The mean, standard deviation, and coefficient of variance (% CV) were calculated for each fatty acid component. Typically, acceptance criteria are met if % CV is less than or equal to 15% for all components.

The results demonstrate that the % CV for each reported fatty acid analytes and fatty acid families was between 0.3-12.8%. Accordingly, within-run precision of the automated, high-throughput process was acceptable, which confirmed and validated the automated, high-throughput process as a viable bioanalytical method.

### Between-Run (Inter-Assay) Precision

Measurements were made from at least three patient sample pools, and all quality control (QC) levels were run in singlicate over twenty runs. Typically, acceptance criteria are met if the % CV is less than or equal to 20% for all components.

The results demonstrate that precision was acceptable for almost all reported fatty acid analytes. Three out of five samples for α-Linolenic acid (ALA) were acceptable, although % CV for ALA in two samples was 35%. However, total precision for ALA was acceptable on all seven instruments used for intra-assay and inter-assay measurements. Moreover, intra-assay precision measurements for ALA were acceptable for all 5 samples. Accordingly, between-run precision of the automated, high-throughput process was acceptable, which confirmed and validated the automated, high-throughput process as a viable bioanalytical method.

### Total Precision for Each Instrument

Total precision test was performed on all seven instruments. Five replicates from three patient sample pools were measured in a single run. This step was then repeated for a second run. The above two steps were repeated for 5 runs over at least three days. Typically, acceptance criteria are met if % CV is less than or equal to 15% for all components.

The results demonstrate that total precision of the automated, high-throughput process using all instruments was acceptable, which confirmed and validated the automated, high-throughput process as a viable bioanalytical method.

### Spike and Recovery/Matrix Effect:

Recovery of an analyte (e.g., fatty acids) in the HDL process is the detector response obtained from a known amount of the analyte added to and extracted from the sample, compared to the detector response obtained for the true concentration of the analyte. Recovery pertains to the extraction efficiency of an analytical method within the limits of variability. Spiking the standard into red blood cells was not practical due to the coagulation caused by mixing hexane-based standard with red blood cells.

Two patient-mixing studies were performed. Two high-level patient samples or pools and two low-level omega-3 patient samples or pools were used to complete these tests. The high-level sample contained greater than 7% omega-3 and the low-level sample contained less than 4% omega-3. The samples were mixed according to the following ratios:

i. High-level Sample >7% omega-3

ii. 3:1 High-level Sample: Low-level Sample

iii. 1:1 High-level Sample: Low-level Sample

iv. 1:3 High-level Sample: Low-level Sample

v. Low-level Sample <4% omega-3

Each level was prepared in triplicate according to steps as described below:
1. The sample was spun down and plasma was removed (1 mL of RBC from the sample required for preparing the triplicates);
2. In test tubes the followings packed PBC were combined:
   i. High-packed RBC tube
   ii. 300 μL high+100 μL low then vortex to mix
   iii. 150 μL high+150 μL low then vortex to mix
   iv. 100 μL high+300 μL low then vortex to mix
   v. Low-packed RBC tube
3. The combined tubes were assayed.
4. Steps 1-3 were repeated with additional high-level and low-level specimens

Typical acceptance criteria are 80-120% mean recovery. The results show that all measured analytes and fatty acid families recovered between 88.6-117.1% with the exception of trans palmitoleic acid, the concentration of which was below detectable limits. Recovery test can show whether a method measures all or only part of the analyte present. Recovery greater than 100% indicates that the method has a degree of error causing over-measurement of the analyte, and is acceptable as long as the recovery rate is within 120%. Accordingly, the spike recover of the automated, high-throughput process was acceptable, which confirmed and validated the automated, high-throughput process as a viable bioanalytical method.

### Linearity and Analytical Sensitivity

A standard curve shows relationship between instrument response and known concentrations of the analyte. When analyte response is identifiable, discrete and reproducible with a precision of 20% and accuracy of 80-120%, the lowest standard on the standard curve is accepted as the limit of quantification.

The lower limit of quantification (LLOQ) was determined as the lowest dilution when it met the 20% precision and 80-120% accuracy. A serial dilution was performed by diluting a sample RBC with saline serially to ×8 dilution (×2, ×4, ×8). The values for percent fatty acids remain the same (<20% CV) at all dilutions except for four analytes (myristic, trans palmitoleic, trans linoleic, and gamma-linolenic), for which peaks became undetectable upon dilution. In standards that were diluted down to the lowest detectable levels, the percent recovery was between 80-120%. All area counts were below 400, which means a result of smaller than 0.1% on any analyte would be produced. All analytes tested in the LLOQ study that reached less than 300 area counts had recoveries within acceptable limits. This verifies that the lowest cut point of 0.1% was adequate for all analytes tested. The results demonstrate that the lowest level of the analytical measurement ranges (AMR) were well down to 0.1% for all analytes, which shows a high analytical sensitivity of the automated, high-throughput process.

### Carryover

Contamination may result from components from a sample that are not distributed from the equipment before being used on a second sample. For example, a fatty acid may remain in the syringe between the movement of a first sample and a second sample. This phenomenon is called carryover. The automated, high-throughput process was analyzed to determine whether carryover was present in the process.

The low standard diluted to the lowest level of quantification from the linearity section, the highest standard, and five double-matrix blanks (saline or blank serum pool without internal standard) were all analyzed for detecting carryover effect for the HDL process. Typically, acceptance criteria are met if the analyte area in all of the five blanks following the highest standard is below the analyte area of the low standard in each run.

The results show that no significant area counts were detected for any analyte measured; thus, running a lower level of detectable dilute standard was not necessary. Accordingly, carryover effect was not present for the automated, high-throughput process.

### Limit of Detection (LOD)

Twenty replicates of saline were run. The mean, standard deviation (SD) and % CV were calculated and the LOD was determined to be the mean+3SD of a blank sample (saline).

The results show that no significant area counts were detected for any analyte. Thus, identifiable peaks below 100 area counts were reported as <0.1%.

What is claimed is:

1. An apparatus comprising:
at least one multi-vessel plate, wherein each vessel is a unit for holding a sample, or mixing and/or reacting a sample with one or more solvents or reagents;
at least one matching multi-cap mat capable of sealing the vessels of the multi-vessel plate during the holding, mixing and/or reacting the sample;
at least one multi-vessel plate holder having sealing units, whereby the multi-vessel plate holder, when the sealing units are engaged, presses the matching multi-cap mat onto the tops of the vessels in the multi-vessel plate sealing the vessels, so as to withstand high pressure and high temperature conditions;
an optional multi-vessel plate heating unit capable of pre-heating to a desirable temperature prior to the introduction of the vessels containing the samples; and
an optional multi-vessel plate separating unit capable of separating one component from the others, if two or more components are present in the vessel in the multi-vessel plate.

2. The apparatus of claim 1, further comprising an automated fluid handler and/or an automated multi-vessel plate handler.

3. The apparatus of claim 1, wherein the lining of the multi-cap mat which contacts the tops of the vessels in the multi-vessel plate is made of a material that does not deteriote and does not contaminate the vessel when heating to the desirable temperature.

4. The apparatus of claim 3, wherein the material is teflon, and the lining of the multi-cap mat has a thickness of 1 to 10 mm.

5. The apparatus of claim 1, wherein the material of the multi-vessel plate heating unit is aluminum.

6. The apparatus of claim 1, wherein the multi-vessel plate heating unit is a multi-vessel plate heating mixer.

7. The apparatus of claim 1, wherein the multi-vessel plate separating unit is a multi-vessel plate centrifuge.

8. The apparatus of claim 1, further comprising:
a labeling equipment for labeling vessels in the multi-vessel plate; and
a label detector.

9. The apparatus of claim 8, wherein the labeling equipment is an automated bar-coding equipment, and the label detector is an automated bar code detector.

10. The apparatus of claim 1, further comprising a multi-vessel plate mixer.

11. The apparatus of claim 10, wherein the multi-vessel plate mixer is a multi-vessel plate vortexer.

12. An apparatus for high-throughput esterification of fatty acids, comprising:
a multi-vessel plate, wherein each vessel is a unit for mixing and/or reacting a sample containing one or more fatty acids with one or more solvents or reagents;
a matching multi-cap mat capable of sealing the vessels of the multi-vessel plate during the mixing and/or reacting the sample;
a multi-vessel plate holder having sealing units, whereby the multi-vessel plate holder, when the sealing units are engaged, presses the matching multi-cap mat onto the tops of the vessels in the multi-vessel plate sealing the vessels, so as to withstand high pressure and high temperature conditions;
a multi-vessel plate heating unit capable of pre-heating to a temperature desirable for esterification of the fatty acids prior to the introduction of the vessels containing the fatty acids; and
a multi-vessel plate separating unit capable of separating the esterified fatty acid from the sample in the vessel of the multi-vessel plate.

13. The apparatus of claim 12, further comprising an automated fluid handler and/or an automated multi-vessel plate handler.

14. The apparatus of claim 12, wherein the lining of the multi-cap mat which contacts the tops of the vessels in the multi-vessel plate is made of a material that does not deteriote and does not contaminate the vessel when heating to the desirable temperature.

15. The apparatus of claim 14, wherein the material is teflon, and the lining of the multi-cap mat has a thickness of 1 to 10 mm.

16. The apparatus of claim 12, wherein the material of the multi-vessel plate heating unit is aluminum.

17. The apparatus of claim 12, wherein the multi-vessel plate heating unit is a multi-vessel plate heating mixer.

18. The apparatus of claim 12, wherein the multi-vessel plate separating unit is a multi-vessel plate centrifuge.

19. The apparatus of claim 12, further comprising:
a labeling equipment for labeling vessels in the multi-vessel plate; and
a label detector.

20. The apparatus of claim 19, wherein the labeling equipment is an automated bar-coding equipment, and the label detector is an automated bar code detector.

21. The apparatus of claim 12, further comprising a multi-vessel plate mixer.

22. The apparatus of claim 21, wherein the multi-vessel plate mixer is a multi-vessel plate vortexer.

23. The apparatus of claim 12, further comprising:
a second multi-vessel plate for holding the separated esterified fatty acids from the plurality of samples;
a second matching multi-cap mat capable of sealing the vessels of the second multi-vessel plate; and
an optional multi-vessel plate holder having sealing units, whereby the multi-vessel plate holder, when the sealing units are engaged, presses the matching multi-cap mat onto the tops of the vessels in the multi-vessel plate sealing the vessels, so as to avoid evaporation and contamination of the samples.

24. The apparatus of claim 12, further comprising a multi-vessel plate measuring unit capable of analyzing the esterified fatty acids.

25. The apparatus of claim 24, wherein the multi-vessel plate measuring unit is a gas or liquid chromatography column.

26. The apparatus of claim 25, wherein the multi-vessel plate measuring unit is a gas or liquid chromatography column with a mass spectrometry detector, an ionization detector or thermal conductivity detector.

27. The apparatus of claim 12, wherein the esterification of the fatty acids is a methyl esterification.

28. The apparatus of claim 27, wherein the pre-heating temperature ranges from 100 to 105° C.

29. A rapid, high-throughput process of analyzing one or more fatty acids in a plurality of samples, comprising:
introducing a plurality of samples containing one or more fatty acids to individual vessels in a multi-vessel plate;
mixing an esterification agent with each sample in the multi-vessel plate to produce esterified fatty acids;
contacting the multi-vessel plate with a multi-vessel plate pre-heated to an esterification temperature of 50 to 300° C., wherein each vessel of the multi-vessel plate is sealed by a matching multi-cap mat;
separating the esterified fatty acids from each sample; and
analyzing the esterified fatty acids from each sample by gas or liquid chromatography.

30. The process of claim 29, wherein at least one of the introducing, mixing, contacting, separating, and analyzing steps are automated, carried out by an automated fluid handler and/or an automated multi-vessel plate handler.

31. The process of claim 29, wherein the mixing of the esterification agent comprises:
adding the esterification agent into each sample in the multi-vessel plate; and
vortexing the mixture in each vessel.

32. The process of claim 29, further comprising:
adding an internal standard to each sample in the multi-vessel plate.

33. The process of claim 32, wherein the internal standard is dissolved in a solvent.

34. The process of claim 29, further comprising:
labeling the plurality of samples in the multi-vessel plate; and
detecting the labeled samples for a sequential processing.

35. The process of claim 34, wherein the labeling step is carried out by an automated bar-coding equipment, and the detecting is carried out by an automated bar code detector.

36. The process of claim 29, wherein the esterification agent is BF₃/methanol.

37. The process of claim 29, wherein the separating step comprises:
mixing an aqueous solvent with each esterified sample in the multi-vessel plate;
simultaneously centrifuging the mixture in each vessel of the multi-vessel plate; and
extracting the organic layer containing the esterified fatty acid from the centrifuged mixture in the multi-vessel plate.

38. The process of claim 29, wherein the analyzing step is carried out by gas or liquid chromatography, and detected by a mass spectrometry, flame ionization detector or a thermal conductivity detector.

39. The process of claim 29, wherein the sample is a blood component selected from the group consisting of red blood cells, whole blood, serum, platelets, white blood cells, plasma, cholesterol esters, triglycerides, free fatty acids, plasma phospholipids, and mixtures thereof.

40. The process of claim 29, wherein the fatty acid component comprises at least one of trans-isomeric unsaturated fatty acid, cis-isomeric monounsaturated fatty acid, Omega-3 fatty acid, Omega-6 fatty acid, and saturated fatty acid.

41. The process of claim 29, wherein the fatty acid component comprises at least one of trans-palmitoleic, trans-oleic, trans-linoleic, cis-palmitoleic, cis-oleic, cis-eicosenoic, cis-nervonic, α-linolenic, eicosapentaenoic, docosapentaenoic, docosahexaenoic, linoleic, γ-linolenic, arachidonic, eicosadienoic, dihomo-γ-linolenic, docasatetraenoic, docosapentaenoic, myristic, palmitic, behenic, lignoceric, arachidic, or stearic acid.

42. The process of claim 41, wherein the fatty acid component comprises trans-palmitoleic, trans-oleic, trans-linoleic, cis-palmitoleic, cis-oleic, cis-eicosenoic, cis-nervonic, α-linolenic, eicosapentaenoic, docosapentaenoic, docosahexaenoic, linoleic, γ-linolenic, arachidonic, eicosadienoic, dihomo-γ-linolenic, docasatetraenoic, docosapentaenoic, myristic, palmitic, behenic, lignoceric, arachidic, or stearic acid.

43. The process of claim 29, wherein the entire step of analyzing by gas or liquid chromatography is carried out in less than about 10 minutes.
